SEQUENCE LISTING (97 sequences)

SEQ ID NO: 1 - DNA, 19 bp, Homo sapiens
cgtccatagcagcgtcccg

SEQ ID NO: 2 - DNA, 18 bp, Homo sapiens
cccgtggtagcagtcgac

SEQ ID NO: 3 - DNA, 19 bp, Homo sapiens
ccttctccggggaactccc

SEQ ID NO: 4 - DNA, 19 bp, Homo sapiens
cgggactcgggctgcagga

SEQ ID NO: 5 - DNA, 19 bp, Homo sapiens
gctttatcctcgggactcg

SEQ ID NO: 6 - DNA, 19 bp, Homo sapiens
gggaaggaggatgaacccc

SEQ ID NO: 7 - DNA, 19 bp, Homo sapiens
gcctgaggactgtggagtg

SEQ ID NO: 8 - DNA, 18 bp, Homo sapiens
ggggaagcctgaggactg

SEQ ID NO: 9 - DNA, 19 bp, Homo sapiens
gagtgaaaatgtcgtccat

SEQ ID NO: 10 - DNA, 19 bp, Homo sapiens
aacggcgactgcgttgccc

SEQ ID NO: 11 - DNA, 19 bp, Homo sapiens
tgttgtccagccacaggcg

SEQ ID NO: 12 - DNA, 19 bp, Homo sapiens
tggttgaggtcgttctccg

SEQ ID NO: 13 - DNA, 19 bp, Homo sapiens
gaagccatgatcgtccccc

SEQ ID NO: 14 - DNA, 19 bp, Homo sapiens
caggcccagtgcaaggggg

SEQ ID NO: 15 - DNA, 19 bp, Homo sapiens
cggccctctcggcaggccc

SEQ ID NO: 16 - DNA, 19 bp, Homo sapiens
ccacagcagagcggccctc

SEQ ID NO: 17 - DNA, 18 bp, Homo sapiens
ccgcatgatcaacatctc

SEQ ID NO: 18 - DNA, 18 bp, Homo sapiens
ttgatccgtgccccccgc

SEQ ID NO: 19 - DNA, 19 bp, Homo sapiens
catccccacggttcattac

SEQ ID NO: 20 - DNA, 19 bp, Homo sapiens
gggggtgtcatccccacgg

SEQ ID NO: 21 - DNA, 19 bp, Homo sapiens
ggctgccagatgcaggggg

SEQ ID NO: 22 - DNA, 19 bp, Homo sapiens
atatcacggtgtccatgac

SEQ ID NO: 23 - DNA, 19 bp, Homo sapiens
actgcaatagcttctgtac

SEQ ID NO: 24 - DNA, 19 bp, Homo sapiens
cactgcattgatgtctgcc

SEQ ID NO: 25 - DNA, 19 bp, Homo sapiens
ggggcacattcccgtgttc

SEQ ID NO: 26 - DNA, 19 bp, Homo sapiens
ccaaaaacaggcatagtgc

SEQ ID NO: 27 - DNA, 19 bp, Homo sapiens
gccacttgatcttggcccc

SEQ ID NO: 28 - DNA, 19 bp, Homo sapiens
catttgccaccaggtcctc

SEQ ID NO: 29 - DNA, 19 bp, Homo sapiens
agatgctgacaagggcccc

SEQ ID NO: 30 - DNA, 19 bp, Homo sapiens
atctctccatacttgttac

SEQ ID NO: 31 - DNA, 19 bp, Homo sapiens
cttggctttgtccacaggc

SEQ ID NO: 32 - DNA, 19 bp, Homo sapiens
aagctctctcaggggtgcc

SEQ ID NO: 33 - DNA, 19 bp, Homo sapiens
tctctgcccgctctcggag

SEQ ID NO: 34 - DNA, 19 bp, Homo sapiens
cggttgagattctggccca

SEQ ID NO: 35 - DNA, 19 bp, Homo sapiens
agaatgtgtccttgtatgg

SEQ ID NO: 36 - DNA, 19 bp, Homo sapiens
gtgcgggtggtccccttcc

SEQ ID NO: 37 - DNA, 19 bp, Homo sapiens
cggggccgagtgcgggtgg

SEQ ID NO: 38 - DNA, 19 bp, Homo sapiens
gggttccatttcggggccg

SEQ ID NO: 39 - DNA, 19 bp, Homo sapiens
ccagagtgtttgttcaggg

SEQ ID NO: 40 - DNA, 19 bp, Homo sapiens
agttaagctgtttgaagtc

SEQ ID NO: 41 - DNA, 19 bp, Homo sapiens
ctcgttgagcttcgtcagg

SEQ ID NO: 42 - DNA, 19 bp, Homo sapiens
tccatagctctccagagtg

SEQ ID NO: 43 - DNA, 19 bp, Homo sapiens
gccctgccagcggcccttc

SEQ ID NO: 44 - DNA, 19 bp, Homo sapiens
gcaccttcacgacaatgtc

SEQ ID NO: 45 - DNA, 19 bp, Homo sapiens
actccagtctcgaaccttc

SEQ ID NO: 46 - DNA, 19 bp, Homo sapiens
aagtccctgctcttccttg

SEQ ID NO: 47 - DNA, 20 bp, Homo sapiens
gccggggacactcttcattg

SEQ ID NO: 48 - DNA, 19 bp, Homo sapiens
tcctgagccggggacactc

SEQ ID NO: 49 - DNA, 19 bp, Homo sapiens
gagcacatttggatgcgag

SEQ ID NO: 50 - DNA, 19 bp, Homo sapiens
ggcaggcacctagcactgg

SEQ ID NO: 51 - DNA, 19 bp, Homo sapiens
atgaggagcaggtggagac

SEQ ID NO: 52 - DNA, 19 bp, Homo sapiens
agtgtgtgatgagagtagg

SEQ ID NO: 53 - DNA, 19 bp, Homo sapiens
agggatccatacggcatcc

SEQ ID NO: 54 - DNA, 19 bp, Homo sapiens
ttcatgtagtacattgtag

SEQ ID NO: 55 - DNA, 19 bp, Homo sapiens
ccacgacgaaattggtgcc

SEQ ID NO: 56 - DNA, 19 bp, Homo sapiens
ttcacagcctggctctggt

SEQ ID NO: 57 - DNA, 19 bp, Homo sapiens
tgccatgtccaaagcaaac

SEQ ID NO: 58 - DNA, 19 bp, Homo sapiens
gtaggaaggccatgcccct

SEQ ID NO: 59 - DNA, 19 bp, Homo sapiens
atgaggggctctagtgtgt

SEQ ID NO: 60 - DNA, 19 bp, Homo sapiens
attgagtgcatgtcgtggg

SEQ ID NO: 61 - DNA, 19 bp, Homo sapiens
caatcattacactacggct

SEQ ID NO: 62 - DNA, 19 bp, Homo sapiens
ttcgggcagtcatgtcctc

SEQ ID NO: 63 - DNA, 19 bp, Homo sapiens
aacttgacatcagccatgc

SEQ ID NO: 64 - DNA, 19 bp, Homo sapiens
accaggacattggaaagag

SEQ ID NO: 65 - DNA, 19 bp, Homo sapiens
aggcaggtgcatacatgcg

SEQ ID NO: 66 - DNA, 19 bp, Homo sapiens
cgggggctacccaggcagg

SEQ ID NO: 67 - DNA, 19 bp, Homo sapiens
agagcttcgggggctaccc

SEQ ID NO: 68 - DNA, 19 bp, Homo sapiens
gtcttcaggcttcttctgc

SEQ ID NO: 69 - DNA, 19 bp, Homo sapiens
tctgctgagcgtctgtttg

SEQ ID NO: 70 - DNA, 20 bp, Homo sapiens
gcactgcaaaactccacatg

SEQ ID NO: 71 - DNA, 19 bp, Homo sapiens
gtgtcaccagttcccacag

SEQ ID NO: 72 - DNA, 19 bp, Homo sapiens
gggtacctcccgtgtcacc

SEQ ID NO: 73 - DNA, 19 bp, Homo sapiens
ccatattggagaggtcagc

SEQ ID NO: 74 - DNA, 19 bp, Homo sapiens
ggccttccaatgccacctt

SEQ ID NO: 75 - DNA, 19 bp, Homo sapiens
ggtgggatggtaggccgaa

SEQ ID NO: 76 - DNA, 19 bp, Homo sapiens
cacatgaggggaaatacct

SEQ ID NO: 77 - DNA, 19 bp, Homo sapiens
catgcagatcttcatgagc

SEQ ID NO: 78 - DNA, 19 bp, Homo sapiens
gggtcgctttgcagggtct

SEQ ID NO: 79 - DNA, 19 bp, Homo sapiens
ggataggcacaatcatgtc

SEQ ID NO: 80 - DNA, 19 bp, Homo sapiens
acttgtcctgcatcttctc

SEQ ID NO: 81 - DNA, 19 bp, Homo sapiens
ggcaaggaccttccagtcc

SEQ ID NO: 82 - DNA, 19 bp, Homo sapiens
cccgacacctctggagttc

SEQ ID NO: 83 - DNA, 19 bp, Homo sapiens
gtgcattcccccaaccatg

SEQ ID NO: 84 - DNA, 18 bp, Homo sapiens
ggggaggtgcattccccc

SEQ ID NO: 85 - DNA, 20 bp, Homo sapiens
ggcctgctgctttggggagg

SEQ ID NO: 86 - DNA, 19 bp, Homo sapiens
gcaaccagaggcctgctgc

SEQ ID NO: 87 - DNA, 19 bp, Homo sapiens
ggaggcgggggaggcaacc

SEQ ID NO: 88 - DNA, 19 bp, Homo sapiens
ccatgactggaggcggggg

SEQ ID NO: 89 - DNA, 19 bp, Homo sapiens
atggaccccaggctggggt

SEQ ID NO: 90 - DNA, 20 bp, Homo sapiens
gggggaaggggatggacccc

SEQ ID NO: 91 - DNA, 20 bp, Homo sapiens
ggtagggatgggggaagggg

SEQ ID NO: 92 - DNA, 20 bp, Homo sapiens
cccgcccctcttgcgcacag

SEQ ID NO: 93 - DNA, 18 bp, Homo sapiens
gctctgagcccgcccctc

SEQ ID NO: 94 - DNA, 20 bp, Homo sapiens
gacaccatgtggcaagtgac

SEQ ID NO: 95 - DNA, 20 bp, Homo sapiens
gctgatccctcccatgttgg

SEQ ID NO: 96 - DNA, 19 bp, Homo sapiens
ggcggggctgatccctccc

SEQ ID NO: 97 - DNA, 19 bp, Homo sapiens
taataaactttattgtgac

(19) United States
(12) Patent Application Publication
(10) Pub. No.: US 2002/0107216 A1
(43) Pub. Date: Aug. 8, 2002

(54) Integrin-linked kinase and its use

(76) Inventors: Shoukat Dedhar, Richmond (CA); Greg Hannigan, Toronto (CA); Arthur Yee, Burnaby (CA)

Correspondence Address: PAMELA J. SHERWOOD, Bozicevic, Field and Francis LLP, Suite 200, 200 Middlefield Road, Menlo Park, CA, 94025, US

(21) Appl. No.: 09/925,548

(22) Filed: Aug. 8, 2001

Related U.S. Application Data
(63) Continuation-in-part of application No. 09/390,425, filed on Sep. 3, 1999.
Application No. 09/390,425 is a continuation of application No. 09/035,706, filed on Mar. 5, 1998.
Application No. 09/035,706 is a continuation-in-part of application No. 08/955,841, filed on Oct. 21, 1997.
Application No. 08/955,841 is a continuation-in-part of application No. 08/752,345, filed on Nov. 19, 1996, now abandoned.
(60) Provisional application No. 60/009,074, filed on Dec. 21, 1995.

(51) Int. Cl. A61K 48/00; C07H 21/04; C12N 9/12; C12N 15/87

(52) U.S. Cl. 514/44; 536/23.2; 435/194; 435/455

(57) ABSTRACT

ILK genetic sequences and methods of use are provided. Antisense oligonucleotides complementary to ILK are useful in downregulating expression for therapeutic and investigative purposes.

### CROSS-REFERENCE TO RELATED APPLICATIONS

This application is a continuation-in-part of U.S. patent application Ser. No. 09/390,425, filed Sep. 3, 1999, which is a continuation of U.S. patent application Ser. No. 09/035,706, filed Mar. 5, 1998, now issued as U.S. Pat. No. 6,001,622, which is a continuation-in-part of U.S. patent application Ser. No. 08/955,841, filed Oct. 21, 1997, now issued as U.S. Pat. No. 6,013,782, which is a continuation-in-part of U.S. patent application Ser. No. 08/752,345, filed Nov. 19, 1996, now abandoned, which claims priority to U.S. Provisional Patent Application No. 60/009,074, filed Dec. 21, 1995, all herein incorporated by reference.

### INTRODUCTION

Research on signal transduction over the years has clearly established the importance of direct, protein-protein interactions in the cytoplasm as a major mechanism underlying the specification of signaling pathways. These interactions can, in part, be those between a receptor and a cytoplasmic protein kinase, or between a protein kinase and its substrate molecule(s). It is known that kinases can form complex signaling cascades, where the activation of one kinase causes it to activate or de-activate another kinase, and so forth through several iterations. One advantage to this type of pathway is that a single “second messenger” can affect a number of different processes, depending on the specific kinase expression pattern in a cell. A particularly interesting second messenger in this respect is phosphatidylinositol 3,4,5 triphosphate [Ptdlns(3,4,5)P₃]. [Ptdlns(3,4,5)P₃] acts on pathways that control cell proliferation, cell survival and metabolic changes—often through protein kinases. This lipid can be produced by PI3 kinases, a family of related proteins (Van haesebroeck et al. (1997) TIBS 22:267; Toker and Cantley (1997) Nature 387:673676). One downstream effector is protein kinase B (PKB/AKT) (Downward (1998) Science 279:673-674). PKB contains a pleckstrin homology (PH) domain, to which the [Ptdins(3,4,5)P₃] signaling molecule binds. In addition, PKB itself is phosphorylated when [Ptdlns(3,4,5)P₃] is present, via two different protein kinases, one of which has been cloned (Stephens et al. (1998) Science 279:710-714; Alessi et al. (1997) Curr. Biol. 7:776). The molecular identity of the other kinase has not been established. The determination of this kinase, as well as its substrates and modulators, is of great interest for providing a point of intervention in this pathway.

If it were determined that a specific kinase regulates integrin function, products that regulate the activity of that kinase could be used for the treatment of cancer, leukemia, solid tumors, chronic inflammatory disease, restenosis, diabetes, neurological disorders, arthritis and osteoporosis, among other indications.

Relevant Literature

A review of integrin mediated signal transduction in oncogenesis may be found in Dedhar (1995) Cancer Metastasis Rev. 14:165-172. Hannigan et al. (1995) 86th Annual Meeting of the American Institute for Cancer Research, provides a brief abstract directed to the cloning of a novel protein kinase associated with beta integrin cytoplasmic tails. Hannigan et al. (1995) Molecular Biology of the Cell suppl. 6, p. 2244, is an abstract directed to the effect of overexpression of a novel integrin linked kinase (ILK) in induction of a transformed phenotype and cyclin D1 expression. Rosales et al. (1995) Biochim Biophys Acta 1242:77-98 reviews signal transduction by cell adhesion receptors. Signaling by cell adhesion receptors may involve aspects that impinge on previously known signaling pathways including the RTK/Ras pathway and serpentine receptor/G protein pathways. A possible signaling role for the Syk tyrosine kinase is described in Lin et al. (1995) J. Biol. Chem. 270:16189-16197.

Miyamoto et al. (1995) Science 267:883-885 compare the roles of receptor occupancy and aggregation on integrin receptor mediation of cell adhesion, signal transduction, and cytoskeletal organization. An EST sequence is provided by EMBL sequence DNA library accession no. p H70160, the Wash. U.-Merck EST project.

The sequences of a number of kinases are known in the art, including human protein kinase B (Coffer and Woodgett (1991) Eur. J. Biochem. 201:475-481). PI3 kinases have been characterized, including phosphatidylinositol 3-kinase gamma polypeptide, (OMIM 601232); phosphatidylinositol 3-kinase alpha polypeptide (OMIM 171834); phosphatidylinositol 3-kinase regulatory subunit (OMIM 171833); mouse PI3 kinase (Genbank M60651); rat PI3 kinase (Genbank D78486, D64045). Glycogen synthase kinase-3 sequences can be accessed at Genbank; the human cDNA sequence has the accession number L40027.

### SUMMARY OF THE INVENTION

Isolated nucleotide compositions and sequences are provided for integrin linked kinase (ILK) genes. Anti-sense oligonucleotides complementary to the ILK coding and/or regulatory sequences are used to down-regulate expression of ILK. The ILK nucleic acid compositions find use in identifying homologous or related genes; for production of the encoded kinase; in producing compositions that modulate the expression or function of its encoded protein; for gene therapy; mapping functional regions of the protein; and in studying associated physiological pathways. In addition, modulation of the gene activity in vivo is used for prophylactic and therapeutic purposes, such as treatment of cancer, identification of cell type based on expression, and the like.

### Description of the Specific Embodiments

Nucleic acid compositions encoding integrin linked kinase (ILK) and complementary anti-sense polynucleotides are provided. The ILK gene product (herein p59_{ILK}) is a serine threonine kinase having two functional domains, the catalytic domain, responsible for phosphotransferase activity (kinase domain), and a non-overlapping domain in the amino terminus, comprised of four contiguous ankyrin-like repeats. Modulation of ILK gene activity in vivo is used for prophylactic and therapeutic purposes. For example, the down-regulation of ILK by administration of anti-sense oligonucleotides is useful as a treatment of cancer, investigation of integrin signaling pathway function, identification of cell type based on expression, and the like. Products that modulate the expression and/or activity of ILK have a therapeutic effect in the treatment of cancer, leukemia, solid tumors, chronic or acute inflammatory disease, restenosis, diabetes, neurological disorders, arthritis and osteoporosis, among other indications.

### Characterization of ILK

The human gene sequence of ILK is provided as SEQ ID NO:1, the encoded polypeptide product as SEQ ID NO:2. The ILK protein is encoded by a 1.8 kilobase pair messenger RNA (1.8 kb mRNA). The sequence of this mRNA was used to deduce the primary amino acid sequence of the protein, which has a predicted molecular weight of 50 kiloDaltons (kDa). The recombinant protein migrates on analytical polyacrylamide electrophoresis gels with an apparent molecular weight of 59 kDa, in rough agreement with the predicted size. The ILK chromosomal locus is mapped to region 11p15.

ILK has novel structural and functional features. The molecular architecture is unusual, in that a protein kinase and an ankyrin repeat domain are contained within the same protein. The kinase domain has a high degree of similarity to other kinase sequences in existing databases, and can be divided into typical subdomains (I through XI) based on this conserved structure. However one amino acid in subdomain VIb of all other protein kinase domains is not present in ILK. Despite this unique structural feature, ILK clearly acts as a protein kinase, and thus represents a prototype member of a new subfamily of protein kinase molecules.

The function of ankyrin repeats in ILK is to mediate protein-protein interactions. The ILK ankyrin repeat domain is not required for the binding of P59_{ILK} to integrin, and it is predicted to mediate the interaction of p59_{ILK} ILK with other cellular protein(s). Thus, p59_{ILK} bridges integrin in the plasma membrane with intracellular proteins active in regulating the cell's response to ECM signals. These proteins are likely to be located in the cytoplasm, or as part of the cell's structural framework (cytoskeleton).

The amino acid sequence of ILK contains a sequence motif found in PH-domains (Klarulund et al. (1997) Science 275:1927-1930). This motif has been shown to be involved in the binding of phosphatidylinositol phosphates (Lemmon et al. (1996) Cell 85:621-624). Amino acids critical to the binding of such lipids to the PH domain are completely conserved in ILK. The phosphatidylinositol 3,4,5, triphosphate binding sites are the lysines at positions 162 and 209 (SEQ ID NO:2). The PH motifs are comprised of residues 158-165 and 208-212 (SEQ ID NO:2). There is a high degree of sequence identity within this motif between ILK and other PH-domain containing proteins such as cytohesin-1 (a β2 integrin cytoplasmic domain interacting protein) and GRP-1. It has been determined that ILK activity is influenced by the presence of phosphatidylinositol 3,4,5, triphosphate, and interacts with other kinase proteins in this pathway.

ILK activity can be stimulated by phosphatidylinositol 3,4,5 trisphosphate in vitro. Both insulin and fibronectin can rapidly stimulate ILK activity in a phosphoinositide-3OH kinase (PI(3)K)-dependent manner. In addition, constitutively active PI(3)K activates ILK. The activated ILK can then inhibit the activity of glycogen synthase kinase-3 (GSK-3), contributing to ILK induced nuclear translocation of β-catenin. ILK can also phosphorylate protein kinase B (PKB/AKT) on serine-473, resulting in its activation, demonstrating that ILK is involved in agonist stimulated PI(3)K-dependent PKB/AKT activation.

In untransformed intestinal epithelial cells, the kinase activity of ILK is inhibited upon cell-extracellular matrix interactions, and overexpression of constitutively active ILK results in anchorage-independent growth and tumorigenicity in nude mice. A consequence of elevation of ILK levels is a disruption of cell-cell interactions and manifestation of fibroblastic cell morphology and phenotypic properties, which include formation of a fibronectin matrix and invasion of collagen gels.

Overexpression of ILK results in a downregulation of E-cadherin expression, formation of a complex between β-catenin and the HMG transcription factor, LEF-1, translocation of β-catenin to the nucleus and transcriptional activation by this LEF-1/β-catenin complex. LEF-1 protein expression is rapidly modulated by cell detachment from the extracellular matrix, and LEF-1 protein levels are constitutively upregulated upon ILK overexpression. These effects are specific for ILK.

Overexpression of ILK stimulates fibronectin matrix assembly in epithelial cells. The integrin-linked kinase activity is involved in transducing signals leading to the up-regulation of fibronectin matrix assembly, as overexpression of a kinase-inactive ILK mutant fails to enhance the matrix assembly. The increase in fibronectin matrix assembly is accompanied by a substantial reduction in cellular E-cadherin. The increased fibronectin matrix assembly is associated with an increased potential for tumor growth in vitro and in vivo.

### Anti-Sense Oligonucleotides

As used herein, the term anti-sense oligonucleotides (ODN) is used to refer to an oligonucleotide molecule or analog thereof that is complementary to at least a portion of the ILK coding sequence, or the untranslated 5′ or 3′ sequences present in ILK mRNA, e.g. an oligonucleotide complementary to the sequence provided in SEQ ID NO:1. Specific examples of oligonucleotide sequences of interest for this purpose may be found in the Examples and SEQ ID NO:13-109.

Antisense molecules are used to down-regulate expression of ILK in cells. The anti-sense reagent may comprise naturally occurring nucleic acids, e.g. RNA or DNA; synthetic ODN having chemical modifications from native nucleic acids; or nucleic acid constructs that express such anti-sense molecules as RNA. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like.

Antisense oligonucleotides can be chemically synthesized by methods known in the art (see Wagner et al. (1993) supra. and Milligan et al., supra.) In one embodiment, the oligonucleotides are chemically modified from the native phosphodiester structure, in order to increase their intracellular stability and binding affinity. A number of such modifications have been described in the literature that alters the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O-5′-S-phosphorothioate, 3′-S′-5-O-phosphorothioate, 3′-CH₂-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5′-methyl-2′-deoxycytidine and 5′-bromo-2′-deoxycytidine for deoxycytidine. 5′-propynyl-2′-deoxyuridine and 5′-propynyl-2′-deoxycytidine have been shown to increase affinity and biological activity when substituted for deoxythymidine and deoxycytidine, respectively.

### ILK Genetic Sequences

Homologs of ILK are identified by any of a number of methods. A fragment of the provided cDNA may be used as a hybridization probe against a cDNA library from the target organism of interest, where low stringency conditions are used. The probe may be a large fragment, or one or more short degenerate primers. Nucleic acids having sequence similarity are detected by hybridization under low stringency conditions, for example, at 50° C. and 10×SSC (0.9 M saline/0.09 M sodium citrate) and remain bound when subjected to washing at 55° C. in 1×SSC. Sequence identity may be determined by hybridization under stringent conditions, for example, at 50° C. or higher and 0.1SSC (9 mM saline/0.9 mM sodium citrate). Nucleic acids that are substantially identical to the provided ILK sequences, e.g. allelic variants, genetically altered versions of the gene, etc., bind to the provided ILK sequences under stringent hybridization conditions. By using probes, particularly labeled probes of DNA sequences, one can isolate homologous or related genes. The source of homologous genes may be any species, e.g. primate species, particularly human; rodents, such as rats and mice, canines, felines, bovines, ovines, equines, yeast, nematodes, etc.

Between mammalian species, e.g. human and mouse, homologs have substantial sequence similarity, i.e. at least 75% sequence identity between nucleotide sequences. Sequence similarity is calculated based on a reference sequence, which may be a subset of a larger sequence, such as a conserved motif, coding region, flanking region, etc. A reference sequence will usually be at least about 18 nucleotides (nt) long, more usually at least about 30 nt long, and may extend to the complete sequence that is being compared. Algorithms for sequence analysis are known in the art, such as BLAST, described in Altschul et al. (1990) J. Mol. Biol. 215:403-10. The sequences provided herein are essential for recognizing ILK related and homologous proteins in database searches.

Nucleic acids encoding ILK may be cDNA or genomic DNA or a fragment thereof. The term ILK gene shall be intended to mean the open reading frame, encoding specific ILK polypeptides, introns, as well as adjacent 5′ and 3′ non-coding nucleotide sequences involved in the regulation of expression, up to about 20 kb beyond the coding region, but possibly further in either direction. The gene may be introduced into an appropriate vector for extrachromosomal maintenance or for integration into a host genome. The term cDNA as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, removed by nuclear RNA splicing, to create a continuous open reading frame encoding an ILK protein.

A genomic sequence of interest comprises the nucleic acid present between the initiation codon and the stop codon, as defined in the listed sequences, including all of the introns that are normally present in a native chromosome. It may further include the 3′ and 5′ untranslated regions found in the mature mRNA. It may further include specific transcriptional and translational regulatory sequences, such as promoters, enhancers, etc., including about 1 kb, but possibly more, of flanking genomic DNA at either the 5′ or 3′ end of the transcribed region. The genomic DNA may be isolated as a fragment of 100 kbp or smaller; and substantially free of flanking chromosomal sequence. The genomic DNA flanking the coding region, either 3′ or 5′, or internal regulatory sequences as sometimes found in introns, contains sequences required for proper tissue and stage specific expression. Sequences of interest also include sequences that cross intron/exon borders, as set forth in SEQ ID NO:12.

The nucleic acid compositions of the subject invention may encode all or a part of an ILK polypeptide. Double or single stranded fragments of the DNA sequence may be obtained by chemically synthesizing oligonucleotides in accordance with conventional methods, by restriction enzyme digestion, by PCR amplification, etc. For the most part, DNA fragments will be of at least 15 nt, usually at least 18 nt or 25 nt, and may be at least about 50 nt. Such small DNA fragments are useful as primers for PCR, hybridization screening probes, etc. Larger DNA fragments, i.e. greater than 100 nt are useful for production of the encoded polypeptide. Regions of the provided sequence that are of interest as fragments include the 5′ end of the gene, i.e. a portion of the sequence set forth in SEQ ID NO:1, nucleotides 1 to 1100.

The ILK genes are isolated and obtained in substantial purity, generally as other than an intact chromosome. Usually, the DNA will be obtained substantially free of other nucleic acid sequences that do not include a ILK sequence or fragment thereof generally being at least about 50%, usually at least about 90% pure and are typically recombinant, i.e. flanked by one or more nucleotides with which it is not normally associated on a naturally occurring chromosome.

### Antisense Modulation of ILK Expression

From a therapeutic point of view, inhibiting ILK activity has a therapeutic effect on a number of proliferative disorders, including inflammation, restenosis, and cancer. Inhibition is achieved in a number of ways. Antisense ILK sequences may be administered to inhibit expression. Where the antisense reagent is a transcribed gene product, an expression vector is used to transcribe the reverse strand (with reference to the normal mRNA coding sequence), where the transcription product may be all or part of the ILK sequence, e.g. as set forth in SEQ ID NO:1. Alternatively, where the antisense reagent is an oligonucleotide, the sequences will be administered in a suitable formulation, as described in detail below.

Expression vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

The gene or ILK protein may be introduced into tissues or host cells by any number of routes, including viral infection, microinjection, or fusion of vesicles. Jet injection may also be used for intramuscular administration, as described by Furth et al. (1992) Anal Biochem 205:365-368. The DNA may be coated onto gold microparticles, and delivered intradermally by a particle bombardment device, or “gene gun” as described in the literature (see, for example, Tang et al. (1992) Nature 356:152-154), where gold microprojectiles are coated with the p59_{ILK} or ILK DNA, then bombarded into skin cells.

The antisense compounds are administered to a subject having undesirable levels of ILK, for example associated with hyperproliferative disorders, e.g. to inhibit tumor growth, to inhibit angiogenesis, to decrease inflammation associated with a lymphoproliferative disorder, to inhibit graft rejection, or neurological damage due to tissue repair, etc. The present compounds are useful for prophylactic or therapeutic purposes. As used herein, the term “treating” is used to refer to both prevention of disease, and treatment of pre-existing conditions. The prevention of proliferation is accomplished by administration of the subject compounds prior to development of overt disease, e.g. to prevent the regrowth of tumors, prevent metastatic growth, diminish restenosis associated with cardiovascular surgery, etc. Alternatively the compounds are used to treat ongoing disease, by stabilizing or improving the clinical symptoms of the patient.

The host, or patient, may be from any mammalian species, e.g. primate sp., particularly humans; rodents, including mice, rats and hamsters; rabbits; equines, bovines, canines, felines; etc. Animal models are of interest for experimental investigations, providing a model for treatment of human disease.

The susceptibility of a particular cell to treatment with the subject compounds may be determined by in vitro testing. Typically a culture of the cell is combined with a subject compound at varying concentrations for a period of time sufficient to allow the active agents to induce cell death or inhibit migration, usually between about one hour and one week. For in vitro testing, cultured cells from a biopsy sample may be used. The viable cells left after treatment are then counted.

The dose will vary depending on the specific compound utilized, specific disorder, patient status, etc. Typically a therapeutic dose will be sufficient to substantially decrease the undesirable cell population in the targeted tissue, while maintaining patient viability. Treatment will generally be continued until there is a substantial reduction, e.g. at least about 50%, decrease in the cell burden, and may be continued until there are essentially none of the undesirable cells detected in the body.

The compounds also find use in the specific inhibition of signaling pathways mediated by ILK. Protein kinases such as ILK are involved in signaling pathways for such important cellular activities as responses to extracellular signals and cell cycle checkpoints. Inhibition of specific protein kinases provides a means of intervening in these signaling pathways, for example to block the effect of an extracellular signal, etc. Defects in the activity of protein kinases are associated with a variety of pathological or clinical conditions, where there is a defect in signaling mediated by protein kinases.

It is also known that many cell types undergo apoptosis if the appropriate contacts with extracellular matrix proteins are not maintained (anoikis). The induction of apoptosis by the subject compounds in such cells predicts an association with the ILK signaling pathway.

There are many disorders associated with a dysregulation of cellular proliferation. The conditions of interest include, but are not limited to, the following conditions.

The subject methods are applied to the treatment of a variety of conditions where there is proliferation and/or migration of smooth muscle cells, and/or inflammatory cells into the intimal layer of a vessel, resulting in restricted blood flow through that vessel, i.e. neointimal occlusive lesions. Occlusive vascular conditions of interest include atherosclerosis, graft coronary vascular disease after transplantation, vein graft stenosis, peri-anastomatic prosthetic graft stenosis, restenosis after angioplasty or stent placement, and the like.

Diseases where there is hyperproliferation and tissue remodelling or repair of reproductive tissue, e.g. uterine, testicular and ovarian carcinomas, endometriosis, squamous and glandular epithelial carcinomas of the cervix, etc. are reduced in cell number by administration of the subject compounds.

Tumor cells are characterized by uncontrolled growth, invasion to surrounding tissues, and metastatic spread to distant sites. Growth and expansion requires an ability not only to proliferate, but also to down-modulate cell death (apoptosis) and activate angiogenesis to produce a tumor neovasculature. Angiogenesis may be inhibited by affecting the cellular ability to interact with the extracellular environment and to migrate, which is an integrin-specific function, or by regulating apoptosis of the endothelial cells. Integrins function in cell-to-cell and cell-to-extracellular matrix (ECM) adhesive interactions and transduce signals from the ECM to the cell interior and vice versa. Since these properties implicate integrin involvement in cell migration, invasion, intra- and extra-vasation, and platelet interaction, a role for integrins in tumor growth and metastasis is obvious. The involvement of integrins with leukocyte homing is also known, and such processes can be modulated by administration of ILK anti-sense reagents.

Tumors of interest for treatment include carcinomas, e.g. colon, duodenal, prostate, breast, melanoma, ductal, hepatic, pancreatic, renal, endometrial, stomach, dysplastic oral mucosa, polyposis, invasive oral cancer, non-small cell lung carcinoma, transitional and squamous cell urinary carcinoma etc.; neurological malignancies, e.g. neuroblastoma, gliomas, etc.; hematological malignancies, e.g. childhood acute leukaemia, non-Hodgkin's lymphomas, chronic lymphocytic leukaemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, etc.; and the like.

Other hyperproliferative diseases of interest relate to epidermal hyperproliferation, tissue remodelling and repair. For example, the chronic skin inflammation of psoriasis is associated with hyperplastic epidermal keratinocytes as well as infiltrating mononuclear cells, including CD4+ memory T cells, neutrophils and macrophages.

The proliferation of immune cells is associated with a number of autoimmune and lymphoproliferative disorders. Diseases of interest include multiple sclerosis, rheumatoid arthritis and insulin dependent diabetes mellitus. Evidence suggests that abnormalities in apoptosis play a part in the pathogenesis of systemic lupus erythematosus (SLE). Other lymphoproliferative conditions include the inherited disorder of lymphocyte apoptosis, which is an autoimmune lymphoproliferative syndrome, as well as a number of leukemias and lymphomas.

### Formulations

The antisense reagents of this invention can be incorporated into a variety of pharmaceutical formulations for therapeutic administration. Particularly, such reagents are formulated for administration to patients for the treatment of ILK dysfunction, where the ILK activity is undesirably high, e.g. to reduce the level of ILK in cancer cells. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal, oral or parenteral. Parenteral administration includes intravenous, intra-arterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Compositions and formulations for oral administration include powders or granules, suspensions or solutions in water or non-aqueous media, capsules, sachets or tablets. Thickeners, flavoring agents, diluents, emulsifiers, dispersing aids or binders may be desirable. Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semi-solids. The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be prepared and formulated as emulsions. Emulsions are typically heterogenous systems of one liquid dispersed in another in the form of droplets usually exceeding 0.1 μm in diameter. Emulsions may contain additional components in addition to the dispersed phases and the active drug that may be present as a solution in either the aqueous phase, oily phase or itself as a separate phase. Pharmaceutical excipients such as emulsifiers, stabilizers, dyes, and anti-oxidants may also be present in emulsions as needed. In one embodiment of the present invention, the compositions of oligonucleotides and nucleic acids are formulated as microemulsions. Typically microemulsions are systems that are prepared by first dispersing an oil in an aqueous surfactant solution and then adding a sufficient amount of a fourth component, generally an intermediate chain-length alcohol to form a transparent system. Microemulsions commonly are prepared via a combination of three to five components that include oil, water, surfactant, cosurfactant and electrolyte.

Other surfactant structures include monolayers, micelles, bilayers and vesicles. As used in the present invention, the term “liposome” means a vesicle composed of amphiphilic lipids arranged in a spherical bilayer or bilayers. Liposomes are unilamellar or multilamellar vesicles that have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo. Liposomes obtained from natural phospholipids are biocompatible and biodegradable; liposomes can incorporate a wide range of water and lipid soluble drugs; liposomes can protect encapsulated drugs in their internal compartments from metabolism and degradation. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act. Liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an antisense RNA. U.S. Pat. No. 5,665,710 to Rahman et al. describes certain methods of encapsulating oligodeoxynucleotides in liposomes. WO 97/04787 to Love et al. discloses liposomes comprising antisense oligonucleotides targeted to the raf gene.

Pharmaceutical carriers or excipients may be liquid or solid and are selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration that do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like. Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives.

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as, for example, dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation. Aqueous suspensions may contain substances that increase the viscosity of the suspension including, for example, sodium carboxymethylcellulose, sorbitol and/or dextran. The suspension may also contain stabilizers.

Certain embodiments of the invention provide pharmaceutical compositions containing (a) one or more antisense compounds and (b) one or more other chemotherapeutic agents which function by a non-antisense mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Other non-antisense chemotherapeutic agents are also within the scope of this invention. Two or more combined compounds may be used together or sequentially. In another related embodiment, compositions of the invention may contain one or more antisense compounds, particularly oligonucleotides, targeted to ILK. Two or more combined compounds may be used together or sequentially.

### Experimental

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXAMPLE 1

### Analysis of ILK in vitro

For analysis of kinase activity in vitro, a bacterially-expressed fusion protein, GST-ILK²³², was SDS-PAGE band purified, and incubated with [γ-32 P]ATP in the presence or absence of the exogenous substrate myelin basic protein. GST-ILK²³² autophosphorylated and labeled MBP efficiently in these assays. Anti-GST-ILK²³² (antibody 91-3) immunoprecipitates of PC3 cell lysates were incubated with [γ-³²P]ATP, similar to experiments performed with purified recombinant GST-ILK²³². ILK immune complexes labeled a protein of apparent Mw of 59 kDa, corresponding to p59_{ILK}, as well as cellular proteins of apparent Mw 32 kDa and 70 kDa, which may be endogenous ILK substrates. Cellular phosphoproteins (serine/threonine) of approximately 32 kDa and 70 kDa, were also seen in β₁ integrin-specific immune complex kinase assays.

In ILK immune complex kinase assays a synthetic peptide representing the β₃ cytoplasmic domain was phosphorylated, while a similar peptide representing the β₃ cytoplasmic domain was not detectably labeled by p59_{ILK}. The β₁ peptide selectively inhibited autophosphorylation of ILK in these reactions, further indicating a differential interaction of the peptides with ILK. The results demonstrating phosphorylation of synthetic β peptides by endogenous ILK are identical to those seen with recombinant GST-ILK²³², and indicate the potential substrate preference of ILK for the β₁ cytoplasmic tail. This does not, however, necessarily rule out an interaction between ILK and the β₃ integrin cytoplasmic domain. Phosphoamino acid analyses of labeled p59_{ILK} and MBP from the immune complex kinase assays detected only phosphoserine in both substrates, as was the case for phosphorylation of these substrates by GST-ILK²³². The β₁ peptide was labeled on serine and threonine residues, with approximately equal stoichiometry. As a control, anti-FAK immune complexes from the same lysates were analyzed for phosphorylation of MBP, and phosphotyrosine was readily detected.

In vitro kinase reactions containing 2 μg of gel-purified GST-ILK²32, with and without 5 μg of myelin basic protein (MBP, Upstate Biotechnologies, Inc.), were analyzed by 10% SDS-PAGE. Immune complexes were generated from PC3 whole cell lysates, using affinity-purified 91-3 antibody. Complexes were assayed for kinase activity, with and without addition of 5 μg/reaction of synthetic peptides, representing β₁ or β₃ integrin cytoplasmic domains or MBP. Products were analyzed by 15% SDS-PAGE (kDa markers at left), and migration of peptides confirmed by Coomassie Blue staining. ³²P-labeled products from the anti-ILK immune complex kinase reactions were isolated and analyzed for phosphoamino acid content. Anti-FAK immune complex kinase assays demonstrated phosphotyrosine on MBP.

Protein kinase assays were performed in 50 μl kinase reaction buffer (50 mM HEPES pH 7.0, 10 mM MnCl₂, 10 mM MgCl₂, 2 mM NaF, 1 mM Na₃ VO₄), containing 10 μCi [γ-32 P]ATP. Reactions were incubated at 30° C. for 20 min, and stopped by the addition of SDS-PAGE sample buffer. For assay of recombinant ILK activity, GST-ILK²³² was adsorbed from bacterial lysates onto glutathione-agarose beads, or GST-ILK²³² was band-purified from 10% SDS-PAGE gels. For immune complex kinase assays, affinity-purified 91-3 anti-ILK antibody was used to generate immunoprecipitates from NP-40 lysates (150 mM NaCl, 1% (v/v) NP-40, 0.5% (w/v) sodium deoxycholate, 50 mM HEPES pH 7.5, 1 μg/ml each leupeptin and aprotinin, 50 μg/ml phenyl-methylsulfonyl flouride) of PC3 cells. Kinase reaction products were resolved on 10-15% SDS-PAGE gels, transferred to PVDF, and phosphoamino acid analysis performed according to a published protocol.

### EXAMPLE 2

### Overexpression of ILK Provides Growth Advantage

The fibronectin-dependent regulation of ILK kinase activity was tested. Plating of rat intestinal epithelial cells, IEC-18, on fibronectin reduced ILK phosphorylation of MBP in immune complex kinase assays, relative to cells plated on plastic or kept in suspension. This fibronectin-dependent reduction of ILK activity was abrogated in IEC-18 cells expressing an activated H-ras allele, indicating that ras transformation disrupts ECM regulation of ILK activity in these cells. An expression vector containing the full-length ILK cDNA, pCMV-ILK, was stably transfected into IEC-18 cells. Twelve stable clones each, of pCMV-ILK and vector control transfectants, were selected and characterized for p59_{ILK} expression levels. Two representative overexpressing subclones, ILK13-A1a3 and -A4a are illustrated. Overexpression of p59_{ILK} disrupted the epithelial morphology of IEC-18 cells. ILK13 clones were more refractile, and grew on LN, FN and VN with a stellate morphology, in marked contrast to the typical, ‘cobble-stone’ morphology of the parental and ILK14 cells. We plated the ILK13-A1a3 and -A4a subclones, the control transfectants, ILK14-A2C3 and -A2C6, and IEC-18 cells, on varying concentrations of the integrin substrates, laminin (LN), fibronectin (FN) and vitronectin (VN). Adhesion of the ILK14 and IEC-18 cells was equivalent, whereas that of the overexpressing subclones was significantly reduced, on all these substrates. Immunoprecipitation analysis indicated that cell surface integrin expression was unaffected. The effect of p59_{ILK} overexpression on anchorage-independent growth was examined by assaying the colony forming ability of ILK transfectants in soft agarose. In marked contrast to IEC-18 and transfectant controls, four independent p59_{ILK} overexpressing subclones, ILK13-A4a, A1a3, and A4C12, formed colonies in these assays. The proliferative rates of all of these clones on tissue culture plastic were equivalent to control rates.

ILK phosphorylation of MBP was assayed in ILK immune complexes, from lysates of IEC-18 intestinal epithelial cells that were harvested from tissue culture plastic and either kept in suspension, or replated on fibronectin for 1 hour. An H-ras-transformed variant of IEC-18, Ras37 (transfected with Rasval12 in pRC/CMV vector), was assayed in parallel. Expression levels of p59_{ILK} in two representative clones of IEC-18 cells, transfected with an ILK expression construct (ILK13), two vector control clones (ILK14), and the parental IEC-18 cells were analyzed. Whole cell RIPA lysates were run out on 10% SDS-PAGE gels, and p59_{ILK} expression analyzed by Western blotting with affinity-purified 92-2 antibody. Representative p59_{ILK} overexpressing clone ILK13-A4a, vector control clone ILK14-A2C3, and parental IEC-18 cells were plated on the ECM substrates LN, FN and VN for 1 hour, then fixed, stained with toluidine blue and photographed (40×mag). d, Adhesion of the ILK overexpressing clones to LN, FN and VN was quantified.

The serial concentrations of ECM showed similar reductions in adhesion of the ILK13 subclones, and ILK14-A2C3 adhesion was identical to that of ILK14-A2C6, on all three substrates. Immunoprecipitation of surface-biotinylated IEC-18, ILK13, and ILK14 subclones, with the anti-FNR and anti-VNR sera, confirmed there was no change in expression of α₅/α₃ β₁ and α₅ β₃/β₅ integrin subunits in the p59_{ILK} overexpressors. Data are representative of two independent experiments. Four ILK13, p59_{ILK} overexpressing clones were plated in soft agarose, and assayed for colony growth after three (experiment 1) and two (experiment 2) weeks. Parent and vector control transfectants were also assayed, and the ras val12 transformed clone, Ras-37, was used as a positive control.

The rat intestinal epithelial cell line IEC-18, and a variant of this line transfected with an activated H-rasval12 allele, expressed from pRC/CMV, were grown on tissue culture plastic in 5% serum-containing medium, washed three times in minimum essential medium (MEM), and harvested with 5 mM EDTA. These were resuspended in 2.5 mg/ml BSA in MEM, and either kept in suspension, or plated on 10 μg/ml fibronectin-coated plates, for 1 hour at 37° C. NP-40 lysates (300 μg) of these cells were immunoprecipitated with affinity-purified 91-3, and immune complex kinase assays (MBP substrate) performed, as described above. IEC-18 were transfected with the expression vector pRC/CMV, containing Plac5 in the forward orientation relative to the CMV promotor. Stable clones were selected in G418, and subcloned through two rounds of limiting dilution. In all, twelve each of ILK and vector control transfectant subclones were isolated. Protein concentrations were determined using the Bradford reagent (Bio-Rad). Two p59_{ILK} overexpressors, ILK13-A1a3 and ILK13-A4a, and two vector transfectant controls, ILK14-A2C3 and -A2C6, were analyzed for effects of ILK overexpression on cell adhesion to ECM substrates. Adhesion was quantified according to published methods. For colony formation assays 3×10⁵ cells were plated in 35 mm wells, in 0.3% agarose, as described previously. Ras-37 were plated at 2×10³/well. Colonies were counted and scored per field (d=1 cm) in duplicate wells, and defined as a minimum aggregate of 50 cells.

These results demonstrate that p59_{ILK} overexpression in the IEC epithelial cells provides a growth advantage, in the absence of proliferative signals normally provided by adhesion.

The transduction of extracellular matrix signals through integrins influences intracellular (‘outside-in’) and extracellular (‘inside-out’) functions, both of which appear to require interaction of integrin cytoplasmic domains with cellular proteins. The association of ILK with β₁ integrin subunits, and specific regulation of its kinase activity by adhesion to fibronectin, suggests that p59_{ILK} is a mediator of integrin signaling. Thus the ankyrin repeat motif likely represents a protein interaction module specifying interactions of ILK with downstream, cytoplasmic or cytoskeletal proteins. Reduced ECM adhesion by the p59_{ILK} overexpressing cells is consistent with our observation of adhesion-dependent inhibition of ILK activity, and suggests that p59_{ILK} plays a role in inside-out integrin signaling. Furthermore the p59_{ILK}-induced, anchorage-independent growth of epithelial cells indicates a role for ILK in mediating intracellular signal transduction by integrins.

### EXAMPLE 3

### The Effect of Anti-ILK on Cell Migration

The role of ILK in cell motility has important implications for normal physiological processes such as inflammation and wound healing, as well as pathological conditions involving tumour invasiveness and metastatic tumour spread, or osteoporosis (bone is essentially an extracellular matrix secreted by osteoblast, or bone-forming cells, and this deposition can be modulated by integrin expression levels and function). Cell motility is a dynamic process that is dependent on integrin-ECM interactions. The “on-off” switch function of protein kinases provides an ideal mechanism for the dynamic regulation of integrin affinity states for ECM substrates. The effect on cell migration of microinjecting highly specific anti-ILK antibodies (thereby inhibiting ILK function) into the cell's cytoplasm is assayed. These effects are assayed in endothelial cells plated on solid substrata, and are extended to include studies on cell migration through three-dimensional gels composed of ECM proteins.

### EXAMPLE 4

### Anti-Sense Oligonucleptides to Inhibit ILK Activity

The sequence of ILK cDNA provides information for the design and generation of synthetic oligonucleotides for “anti-sense” inhibition of ILK activity. This term derives from the strategy of employing a reverse complement of the coding, or sense strand of a specific messenger RNA, known as an anti-sense oligonucleotide (AO). By binding to its complementary mRNA, the AO inhibits translation of that mRNA into protein, thereby preventing normal protein accumulation in the cell. ILK AO derived from the ILK mRNA sequence closest to the presumptive translational start site is tested, as this is predicted to provide the most successful reagent.

Regardless of the actual chemistry used to construct the AO, or modifications to an anti-ILK AO to improve its efficiency, the cDNA sequence of ILK provides the information for derivation of a specific AO. The cDNA sequence of ILK is also used to design oligonucleotide reagents, known as degenerate primers (due to the degeneracy of the genetic code), for use in polymerase chain reaction (PCR)-based screens for cDNAs structurally related to ILK. Similarly, the ILK cDNA is used to screen for related genes in a more conventional screen of genomic or cDNA libraries, by employing less stringent (i.e. milder) hybridization conditions during screening. In this way, distinct cDNA or DNA sequences significantly related to ILK (>50% nucleotide identity) can be isolated, and a family of ILK-related kinases identified in a non-random fashion.

### EXAMPLE 5

### Mapping of ILK Chromosomal Locus to Assess Imprinted Copies of Gene

High resolution mapping of the ILK chromosomal locus through fluorescent in situ hybridization (FISH) to metaphase (i.e. separated and identifiable) human chromosomes has placed the ILK gene on chromosome 11p15. FISH is known to those skilled in the art. High resolution mapping uses known marker genes in this region. Certain genes (e.g. insulin-like growth factor 2, IGF2) in the 11p15 region have been shown to be imprinted (i.e. preferentially expressed from either the maternally or paternally-derived chromosomes). This imprinting effectively provides a functional deletion or “knock-out” of one of the two inherited copies of a gene. Thus, mutation of the non-imprinted allele (copy) has a more profound outcome, since no compensatory activity is available from the imprinted allele. Also, 11p15 has been identified as a region subject to loss-of-heterozygosity, or LOH, in a subset of breast tumour patients. LOH results in the loss of one allele, for example by gene deletion, and is a mechanism underlying the contribution of a number of tumor suppressor genes to the development of various cancers (e.g. BRCA1 in breast, DCC in colon carcinoma, and RB1 in retinoblastoma). Thus ILK cDNA sequence is used to develop DNA reagents for the diagnosis and prognostic indications of a significant subset of breast cancers, and these reagents contribute to the molecular classification of such tumors. As mentioned above, the gene(s) on 11p15 contributing to some inherited cases of long QT syndrome are identified, and the candidacy of ILK as a causative gene for this cardiac condition, are evaluated by looking for alterations in ILK gene structure in families where 11p15 associations have been made.

### EXAMPLE 6

### Induction of in vivo Tumorigenesis by Overexpression of ILK

Overexpression of ILK down-regulates E-cadherin, which is an important epithelial cell adhesion molecule mediating cell-cell communication/interaction. The loss of E-cadherin induced by overexpression of ILK in epithelial cells suggests that ILK may promote tumorigenicity in vivo. To test this, we injected cells expressing varying levels of ILK into athymic nude mice subcutaneously. Mice were inoculated subcutaneously with the cells expressing high (ILK13-A1a3 and A4a) or low (IEC-18 and ILK14-A2C3) levels of ILK (10⁷ cells/mouse in PBS). The mice were monitored for tumor formation at the site of inoculation after three weeks. Tumors arose within three weeks in 50% to 100% of the mice injected with the ILK13 cells (10⁷ cells/mouse) that overexpress ILK, whereas no tumors were detected in the mice that were injected with the same number of the IEC-18 or ILK14 cells expressing lower levels of ILK (Table 1). Thus, overexpression of ILK in these epithelial cells promotes tumor formation in vivo.

TABLE 1
Tumorigenicity of ILK Overexpressing IEC-18 Cells

| Cell Line | Number of Mice with Tumors at 3 weeks |
|---|---|
| IEC-18 | 0/6 |
| ILK14-A2C3 | 0/6 |
| ILK13-A1a3 | 6/6 |
| ILK13-A4a | 3/6 |

### EXAMPLE 7

### Increased Expression of ILK in Human Breast Carcinoma

The expression of Integrin Linked Kinase in human breast carcinomas was determined by immunohistochemical staining of paraffin embedded sections from human breast cancer biopsies. Affinity purified anti-ILK polyclonal antibody was used followed by conjugated secondary antibody. The positive staining observed was completely abolished by absorption of the antibody to ILK-coupled sepharose beads. A total of 30 samples have been examined so far. In every case ILK expression levels are markedly elevated in tumor tissue compared to normal ducts and lobules. A normal region shows well formed ducts with a single layer of epithelial cells. ILK staining is most prominent in epithelial cells. The stroma appears negative. In ductal carcinoma in situ (DCIS), multiple cell layers are present with markedly elevated ILK staining in the tumor cells. In invasive carcinomas there is markedly elevated expression of ILK compared to the normal tissue.

### EXAMPLE 8

### Regulation of LEF-1 Expression and Complex Formation

Overexpression of ILK results in a downregulation of E-cadherin expression, formation of a complex between β-catenin and the HMG transcription factor, LEF-1, translocation of β-catenin to the nucleus, and transcriptional activation by this LEF-1/β-catenin complex. LEF-1 protein expression is rapidly modulated by cell detachment from the extracellular matrix and LEF-1 protein levels are constitutively upregulated upon ILK overexpression. These effects are specific for ILK, since transformation by activated H-ras or v-src oncogenes do not result in the activation of LEF-1/β-catenin. The results demonstrate that the oncogenic properties of ILK involve activation of the LEF-1/β-catenin signaling pathway via elevation of LEF-1 expression.

Overexpression of ILK in rat intestinal epithelial cells (IEC-18) induces a loss of epithelial morphology, characterized by a disruption of cell-cell adhesion and the acquisition of a fibroblastic morphology that includes enhanced fibronectin matrix assembly. This altered morphology is accompanied by the ability of the cells to progress through the cell cycle in an anchorage-independent manner and to form tumors in nude mice. To determine whether the loss of cell-cell adhesion was accompanied by an increased invasive phenotype, the invasiveness of IEC-18 parental cells and ILK-overexpressing (ILK-13) cells was tested in a collagen gel invasiveness assay. The data is shown in Table 2.

The ILK-13 cells are much more invasive than the parental and control transfected (ILK-14) cells that have been transfected with an ILK anti-sense cDNA construct. Collagen-gel invasion by epithelial cells is normally associated with an epithelial to mesenchymal transformation characterized by the down regulation of E-cadherin expression. Notably, the expression of E-cadherin protein is completely lost in ILK overexpressing cells (ILK-13), but is maintained in control transfected cells, reduced in IEC-18 cells transfected with activated H-ras cDNA, and greatly reduced in v-src transformed cells. In contrast, the steady-state levels of the expression of the intracellular E-cadherin binding protein, β-catenin, is unchanged by ILK overexpression and is similar in all IEC cell transfectants.

The subcellular localization of β-catenin was examined in these cells. In sharp contrast to the localization of β-catenin at the plasma membrane and at cell-cell adhesion sites in the parental IEC-18 and control transfected cell clones (A2c3 and A2c6), β-catenin is localized entirely in the nuclei of ILK overexpressing ILK-13 clones (A4a, A1a3). This ILK-induced nuclear localization of β-catenin is dependent on an active kinase, since overexpression of a kinase-deficient ILK (E359K) did not induce nuclear translocation of β-catenin which remains localized largely to the plasma membrane. Likewise, overexpression of kinase-deficient ILK also did not result in a loss of E-cadherin expression. The translocation of β-catenin to the nucleus is a specific property of ILK, since in IEC-18 cells transfected with activated H-ras or v-src oncogenes, β-catenin is not translocated to the nucleus, but is either localized to the plasma membrane or is expressed diffusely in the cytoplasm. Although these oncogenes also disrupt the epithelial morphology of IEC-18 cells and result in the downregulation of E-cadherin expression, the translocation of β-catenin to the nucleus is a property unique to ILK expression, suggesting that loss of E-cadherin expression and β-catenin nuclear translocation may be regulated differentially. Overexpression of ILK in mouse mammary epithelial cells also results in similar alterations in the phenotypic properties described above for the IEC-18 cells.

Translocation of β-catenin to the nucleus can be induced by the activation of the Wnt signaling pathway, which initially results in an elevation of free cytosolic β-catenin due to decreased degradation. Alternatively, loss of expression or mutations in the tumor suppressor protein APC and certain mutations in the β-catenin gene lead to cytosolic β-catenin stabilization and nuclear translocation. The nuclear translocation of β-catenin is associated with complex formation between β-catenin and members of the HMG transcription factors, LEF-1/TCF which then activate (or silence) transcription of target genes. Since the steady state levels of β-catenin were not changed by ILK overexpression “uncomplexed” β-catenin levels were measured, as determined by binding to a cytoplasmic domain peptide of E-cadherin. “Uncomplexed ” pools of β-catenin in ILK overexpressing clones were found to be low and unaltered compared to IEC-18 cells or control ILK 14 clones. This indicates that most β-catenin is likely complexed with nuclear components such as transcription factors and DNA. In contrast, free P-catenin pools in Ras and Src transformed cells were high, consistent with decreased E-cadherin expression and indicating disruption of E-cadherin-β-catenin interaction. However, the increased free pools of β-catenin did not result in nuclear translocation of β-catenin.

The expression levels of LEF-1, a member of the family of HMG transcription factors that bind β-catenin, were measured. The expression of LEF-1 is dramatically higher in six independent ILK expressing ILK-13 cell clones as compared with six independent control transfected ILK-14 clones, as well as 2 activated H-ras transfected and v-src transfected IEC-18 clones. E-cadherin expression is lost in all 6 ILK-13 cell lines. Transient induction of ILK expression using an ecdysone inducible ILK construct also resulted in an increase of LEF-1 expression. As expected, the increased levels of LEF-1 and the nuclear translocation of β-catenin are associated with enhanced complex formation between LEF-1 and β-catenin in the ILK overexpressing cells.

LEF-1 is a transcription factor that is by itself, unable to stimulate transcription from multimerized binding sites, however in association with β-catenin, LEF1/TCF proteins can augment promoter activity from multimerized binding sites. Transcriptional activation from a TCF/β-catenin responsive promoter construct was examined in ILK-overexpressing cells and control kinase-deficient ILK expressing cells. High promoter activity was observed in ILK-overexpressing cells and the extent of transcriptional activation was reduced with promoter constructs containing mutations in the multimerized LEF-1/TCF binding sites. Moreover, nuclear extracts were analyzed from ILK-overexpressing cell clones and from cell clones transfected with an anti-sense or kinase-deficient ILK cDNA to identify proteins that bind the LEF/TCF binding site. The abundance of a nuclear factor in ILK-overexpressing cells that displays the same binding site specificity, immunoreactivity and electrophoretic mobility as LEF-1, was found to be markedly enhanced relative to the unrelated DNA-binding protein Oct-1.

ILK binds to the cytoplasmic domain of β₁ and α₃ integrin subunits, and its kinase activity is downregulated upon cell adhesion to extracellular matrix (ECM) proteins. Overexpression of constitutively activated ILK overcomes this regulation of ILK activity by integrin occupation and results in decreased cell adhesion to ECM-protein. Cell adhesion to ECM suppresses LEF-1 expression, which is rapidly, but transiently, elevated upon cell detachment in ILK-14 and ILK13 cells. However in ILK overexpressing ILK-13 cells the elevation in LEF-1 levels are more robust and are maintained at high levels for as long as 16 hours in suspension. Furthermore, LEF-1 levels are also higher in adherent ILK-13 cells compared to ILK-14 cells.

These data indicate that ILK overexpression overcomes the regulation of LEF-1 expression by adhesion-deadhesion, and that the maintenance of constitutively high levels of LEF-1 result in enhanced complex formation between LEF-1 and β-catenin, translocation of β-catenin to the nucleus, and transcriptional activation of responsive genes. Since TCF/β-catenin has been shown to induce transcription of genes encoding homeobox proteins that regulate mesenchymal genes e.g. Siamois in Drosophila, this pathway is likely to mediate the observed epithelial to mesenchymal transformation, as well as the oncogenic properties of ILK in these intestinal epithelial cells, since constitutive activation of TCF/β-catenin is oncogenic in human colon carcinomas. The data presented here also suggest a connection between the expression of E-cadherin and the signaling properties of β-catenin in mesenchymal induction in ILK transformed cells, in agreement with the work of others that E-cadherin can antagonize β-catenin signaling, although the loss of E-cadherin expression does not always correlate with nuclear β-catenin translocation e.g. in the v-src transformed cells.

An additional pathway is demonstrated to that by activated Wnt-1 leading to increased LEF-1/β-catenin complex formation and transcriptional activation. These data also corroborate previous work showing that overexpression of LEF-1 can work independently of Wnt to enhance LEF-1-β-catenin complex induced transcription. Here it is shown that in contrast to the effects of Wnt-1, activated ILK can dramatically induce the formation and nuclear translocation of LEF-1/β-catenin complexes without a corresponding increase in the free pool of β-catenin. This ILK-regulated pathway may be modulated via cell adhesion to ECM, but can be constitutively activated by ILK overexpression.

Methods

Cells and cell culture. IEC-18 rat intestinal epithelial cells were stably transfected with a mammalian vector incorporating ILK to produce clones overexpressing wt ILK in the sense orientation (ILK-13) or antisense orientation (ILK-14), or to produce a kinase-deficient form of ILK (IEC-18GH31RH) described below. IEC-18 cells were also stably transfected to overexpress H-ras (Ras 33, Ras 37) (Buick et al. (1987) Exp. Cell. Res. 170:300-309), and v-src (Src2, Src4) (Filmus et al. (1988) Mol. Cell. Biol. 8:4243-4249). Cells were grown in d-MEM containing 5% FCS, 2 mm L-glutamine, glucose (3.6 mg/ml), insulin (10 μg/ml), and G418 (40 μg/ml) was added to transfected cells to maintain selection pressure.

Site directed mutagenesis of ILK kinase domain. Mutations were introduced into wt ILK-cDNA with the Promega Altered Sites II System (Promega, Madison, Wis.). Mutant oligomers (with the altered nucleotide underlined) were used to change lysine at position 220 to an arginine (K220R, (SEQ ID NO:9) 5′ CCTTCAGCACCCTCACGACAATGTCATTGCCC 3′) and glutamic acid at position 359 to lysine (E359K, (SEQ ID NO:10) 5′ CTGCAGAGCTTTGGGGGCTACCCAGGCAGGTG 3′). Mutant clones were confirmed by dideoxy sequencing and subcloned into pGEX4T-1 GST fusion vector (Pharmacia, Piscataway N.J.) to express GST-ILK in E. coli (BL21-DE3) and into pcDNA3 (Invitrogen, San Diego, Calif.) to stably transfect kinase-deficient ILK into the IEC-18 cell line (IEC-18GH31RH containing the E359K mutation).

Inducible expression of ILK. Full length wt ILK cDNA (1.8 Kb) was subcloned into the Ecdysone-inducible expression vector pIND (Invitrogen, San Diego, Calif.) and 10 μg were transiently co-transfected with 10 μg of the complementary regulator vector pVgRXR into subconfluent cells growing in 6 well plates with 20 μl of Lipofectin (Gibro-BRL, Gaithersburg, Md.). ILK expression was induced 6 hrs later with the addition of 1 μM muristerone A.

Western blotting and immunoprecipitation. Cells were lysed for 10 minutes on ice in NP-40 lysis buffer (1% NP40, 50 mM Hepes, pH 7.4, 150 mM NaCl, 2 mM EDTA, 2 mM PMSF, 1 mM Na-o-vanadate, 1 mM NaF, 10 μg/ml aprotinin, 10 μg/ml leupeptin). Extracts were centrifuged with the resulting supernatants being the cell lysate used in assays. Lysates were electrophoresed through SDS-PAGE and transferred to Immobilon-P membranes (Millipore, Bedford, Md.). Antibodies used to probe Western blots were: rabbit anti-ILK, monoclonal anti-E-cadherin and monoclonal anti-β-catenin (Transduction Labs, Lexington, Ky.), and rabbit anti-LEF-1 (Travis et al. (1991) Genes & Development 5:880-894). Bands were visualized with ECL chemiluminescent substrate (Amersham, Buckinghamshire, England). For immunoprecipitation, NP-40 lysates were rotated with primary antibody ON at 4° C., then rotated with Protein G-Sepharose (Pharmacia, Uppsala, Sweden) for 2 hrs at RT. Beads were pelleted, boiled in electrophoresis sample buffer (non-reducing), centrifuged and supernatants were electrophoresed. Protein concentrations were determined by the Bradford assay (Bio-Rad, Hercules, Calif.).

Invasion assay. Confluent cells were trypsinized and 7.5×10⁴ cells in 1.5 ml of complete medium were seeded onto 1.5 ml of a three dimensional collagen gel in a 35 mm tissue culture dish (Montesano et al. (1985) Cell 42:469-477). Upon reaching confluence (3 days), the cultures were incubated for a further 4 days, then fixed in situ with 2.5% glutaraldehyde in 100 mM cacodylate buffer (pH 7.4), and photographed at different planes of focus. Invasion was quantitated by counting the number of cells that had migrated below the surface of the collagen gel. Five randomly selected fields measuring 1.0 mm×1.4 mm were photographed at a single level beneath the surface monolayer using a 10×phase contract objective.

Indirect immunofluorescence. Cells were grown on cover slips, washed with PBS, fixed in 4% paraformaldehyde in PBS for 12 minutes, washed with PBS, permeabilized in 0.1% Triton X-100 in PBS for 10 minutes, blocked with 4% BSA in PBS for 30 minutes at RT, incubated with rabbit anti-β-catenin (Hulsken et al. (1994) J. Cell Biol. 127:2061-2069) diluted 1:400 in 0.1% Triton X-100 for 60 minutes at 37° C., washed with PBS, incubated with rhodamine conjugated goat anti-rabbit IgG (Jackson ImmunoResearch, West Grove, Pa.) diluted 1:50 in 0.1% Triton X-100 for 30 minutes at 37° C., washed with PBS, then mounted onto a slide with Slow-Fade Antifade (Molecular Probes Inc., Eugene, Oreg.). Cells were viewed at 100 fold magnification using a Zeiss Axiovert 135 fluorescence microscope.

Reporter gene assay. Cells were transiently transfected with 0.3 μg of a luciferase reporter gene construct containing a series of optimal or mutated LEF-1/TCF binding sites (Korinek et al. (1997) Science 275:1784-1787), along with 0.05 μg of a CAT gene construct containing a ribosomal promoter (Hariharan et al. (1989) Genes & Development 3:1789-1800) to control for transfection efficiency. Extracts were prepared and assayed 48 hours after transfection.

Electrophoretic mobilty shift assay. Twenty μg of nuclear extract were incubated with 1 fmole of ³2 P-labeled duplex oligonucleotide probe specific for LEF-1, in 20 μl of binding buffer containing 200 ng poly[d(I-C)], 400 ng salmon sperm DNA, and electrophoresed through a 5% native polyacrylamide gel (Travis et al. (1991) Genes and Development 5:880-894). For DNA competition, an 800-fold molar excess of oligonucleotide containing a specific LEF-1 binding site or a non-specific EBF-binding site (Hagman et al. (1991) E.M.B.O. J. 10:3409-3417) was included in the DNA-binding reaction. For antibody addition, 1 μl of polyclonal anti-LEF-1 antibody or 1 μl of monoclonal anti-β-catenin antibody (Transduction Labs, Lexington, KY) were used.

TABLE 2
INVASION OF COLLAGEN GELS

| Cell Line | Invading cells/field |
|---|---|
| IEC-18 | 10 +/− 0.87 |
| ILK14/A2c | 67.8 +/− 1.32 |
| ILK13/A1a | 326.73 +/− 2.61 |
| ILK-13/A4a | 83.6 +/− 4.68 |

After seeding 7.5×10⁴ cells, the number of invading cells in 5 photographic fields from 3 separate experiments (total of 15 fields/cell line) were counted. Results are given as the mean number of invading cells +/−SEM. *p<<0.01 between ILK13/A1a3 compared to IEC-18 and ILK-14 cells (Students unpaired t=test).

### EXAMPLE 9

### Regulation of Fibronectin Matrix Assembly, E-cadherin Expression and Tumorigenicity

A common feature of many oncogenically transformed cells is that they lose the ability of assembling a fibronectin (Fn) matrix. However, exceptions to the rule of neoplastic cells lacking Fn matrix clearly exist. For example, Fn matrix assembly is dramatically enhanced in hairy cell leukemia cells. The specific phenotype (inhibition or stimulation of Fn matrix assembly) is probably determined by the origin of the neoplastic cells and the initial target of the oncogenic transformation. Because Fn matrix has a major impact on cell adhesion, migration, cell growth and cell differentiation, an understanding of the molecular mechanism by which cells control Fn matrix assembly may provide important information on tumorigenicity and may lead to new ways of controlling tumor growth.

Binding of Fn by specific integrins is critical in initiating Fn matrix assembly. Fn fragments containing the RGD-containing integrin binding site or antibodies recognizing the integrin binding site inhibit Fn matrix assembly. In addition, antibodies to α₅ β₁ integrin reduce the deposition of Fn into extracellular matrix by fibroblasts. In addition to α₅ β₁ integrin, members of the β₃ integrins (α.sub.IIb β₃ and α.sub.v β₃) also initiate Fn matrix assembly, although some of the other Fn binding integrins such as α₄ β₁ or α.sub.v β₁ do not. The ability of cells to use multiple integrins to support Fn matrix assembly provides the cells with a versatile mechanism for control of Fn matrix assembly. It may also explain why certain cells, such as fibroblastic cells derived from α₅ integrin null mutant embryos, assemble a Fn matrix in the absence of α₅ β₁. The primary role of α₅ β₁ in Fn matrix assembly appears to involve initiating the assembly, as Fn mutants lacking the α₅ β₁ integrin binding site could not be assembled into Fn matrix unless in the presence of native Fn.

Activation of specific Fn binding integrins, either by mutations at the integrin cytoplasmic domains or using activating antibodies, dramatically stimulates Fn matrix assembly. The ability of a cell to assemble a Fn matrix is not only controlled by the types of integrins it expresses but also regulated by the Fn binding activity of the integrins. The extracellular ligand binding affinity of integrins can be controlled from within the cells (inside-out signaling).

Integrin-linked kinase (ILK) may be involved in regulating Fn matrix assembly. ILK binds to the cytoplasmic domains of both β₁ and β₃ integrins, and phosphorylates the β₁ cytoplasmic domain in vitro. Overexpression of ILK in epithelial cells dramatically stimulated integrin-mediated Fn matrix assembly, down-regulated E-cadherin, and induced tumor formation in vivo. The data identify ILK as an important regulator of pericellular Fn matrix assembly, and suggest a critical role of this integrin-linked kinase in cell-cell interactions and tumorigenesis.

Reagents

All organic chemicals were of analytic grade and were obtained from Sigma Chemical Co. (St. Louis, Mo.) or Fisher Scientific Co. (Pittsburgh, Pa.) unless otherwise specified. Media for cell culture were from Gibco Laboratories (Grand Island, N.Y.). Fetal bovine serum was from HyClone Laboratories, Inc. (Logan, Utah). Polyclonal rabbit anti-α₅ integrin cytoplasmic domain antibody AB47 was generated using a synthetic peptide representing the membrane distal region of the α₅ integrin cytoplasmic domain ((SEQ ID NO:11) LPYGTAMEKAQLKPPATSDA). Polyclonal rabbit anti-Fn antibody MC54 was raised against purified plasma Fn and purified with a protein A-Sepharose affinity column (Wu et al. (1993) J. Biol. Chem. 268:21883-21888). Polyclonal rabbit anti-29 kDa fragment of Fn antibody was raised against the aminoterminal 29 kDa fragment of Fn and was further purified using Sepharose beads coupled with the 29 kDa fragment of Fn (Limper et al. (1991) J. Biol. Chem. 266:9697-9702). Anti-ILK polyclonal antibody 91-4 was prepared in rabbits as described previously (Hannigan et al. (1996) Nature 379:91-96). Monoclonal hamster anti-rat α5 integrin antibody (HMα5-1) and mouse anti-rat β₃ integrin antibody (F11) were from PharMingen (San Diego, Calif.). Monoclonal mouse anti-vinculin antibody (hVIN-1) and purified rabbit IgG were purchased from Sigma (St. Louis, Mo.). The Fn fragments (110 kDa RGD containing integrin binding fragment, the 20 kDa and 70 kDa amino terminal fragments, and the 60 kDa gelatin binding were prepared as previously described (Quade and McDonald (1988) J. Biol. Chem. 263:19602-19609). cDNA Vectors, Transfection and Cell Culture. Rat intestinal epithelial cells (IEC-18) were maintained in α-MEM medium (Gibco Laboratories, Grand Island, N.Y.) supplemented with 5% FBS (Atlanta Biologicals, Norcross, Ga.), 3.6 mg/ml glucose, 10 μg/ml insulin and 2 mM glutamine. The pRC/CMV and metallothionein promoter (MT) driven expression vectors containing sense and anti-sense full length ILK cDNA sequences were generated as described above. The expression vectors were transfected into IEC-18 cells using calcium phosphate and the transfected cells were selected with G418 as described. The expression of human ILK in IEC-18 cells transfected with the MT-ILK expression vectors (MT-ILK) was induced by growing the cells in α-MEM medium containing 125 μM ZnSO₄ and 2.5 μM CdCl₂ for 24 to 48 hours. The kinase-inactive ILK mutant (GH31R) was generated by a single point mutation (E.fwdarw.K) at amino acid residue 359 within the kinase subdomain VIII using the Promega Altered Site II in vitro Mutagenesis System. The mutated DNA was cloned into a pGEX expression system (Pharmacia), and expressed as a GST fusion protein. Kinase assays were carried out using the recombinant protein as described above and the results showed that the E³59 .fwdarw.K point mutation completely inactivated the kinase activity. The cDNA encoding the kinase-inactive mutant was cloned into a pcDNA3 expression vector (Invitrogen), transfected into IEC-18 cells and stable transfectants were selected.

Determination of ILK, E-cadherin and β₁ integrin levels. The cellular levels of ILK and E-cadherin were determined by immunoblot using an affinity-purified polyclonal rabbit anti-ILK antibody 91-4, and an anti-E-cadherin antibody (Upstate Biotechnologies, Inc.). The cell surface expression of α₅ β₁ integrins was estimated by immunoprecipitation of surface biotinylated cell lysates with a polyclonal rabbit anti-α₅ β₁ antibody.

Immunofluorescent Staining. Fn matrix assembly was analyzed by immunofluorescent staining of cell monolayers (Wu et al. (1995) Cell 83:715-724). Cells were suspended in the α-MEM medium containing 5% FBS and other additives as specified in each experiment. Cells were plated in 12-well HTC.sup.R slides (Cel-Line, Inc., Newfield, N.J.; 50 μl/well) at a final density of 2×10⁵ cells/ml and cultured in a 37° C. incubator under a 5% CO₂-95% air atmosphere. Cells were fixed with 3.7% paraformaldehyde, and staining with the polyclonal rabbit anti-Fn antibody MC54 (20 μg/ml) and Cy3-conjugated goat anti-rabbit IgG antibodies (Jackson ImmunoResearch Lab, Inc, West Grove, Pa.; 2.5 μg/ml). Stained cell monolayers were observed using a Nikon FXA epifluorescence microscope and representative fields were photographed using Kodak T-Max 400 or Ektachrome 1600 direct positive slide film. To obtain representative images, exposure times for different experimental conditions were fixed, using the positive, e.g., matrix forming cells, as the index exposure length.

In double staining experiments, 3.7% paraformaldehyde fixed cells were permeablized with 0.1% Triton X-100 in TBS containing 1 mg/ml BSA. The cells were then incubated with primary antibodies from different species as specified in each experiment. After rinsing, the bound primary antibodies were detected with species-specific Cy3- and FITC-conjugated secondary antibodies. Stained cell monolayers were observed using a Nikon FXA epifluorescence microscope equipped with Cy3 and FITC filters.

For inhibition studies, ILK13-A4a cells that overexpress ILK were plated in 12-well HTC.sup.R slides in the α-MEM medium containing 5% FBS and other additives as specified (2 μM anti-29 kDa Fn fragment antibody, 2 μM rabbit control IgG, or 4.2 μM of one of the following Fn fragments: 110 kDa RGD containing integrin binding fragment of Fn, 70 kDa aminoterminal fragment of Fn or 60 kDa gelatin binding fragment of Fn). The cells were cultured for four hours, and then fixed and stained with the polyclonal rabbit anti-Fn antibody and the Cy3-conjugated goat anti-rabbit IgG antibodies as described above.

Isolation and Biochemical Characterization of Extracellular Matrix Fn. To isolate and biochemically characterize extracellular matrix Fn, the cells were cultured in 100 mm tissue culture plates (Corning, Inc., Corning, N.Y.) in α-MEM medium supplemented with 5% FBS, 2 mM L-glutamine, 3.6 mg/ml glucose, 10 μg/ml insulin and other additives as specified in each experiment for two days. Then the cell monolayers were washed three times with PBS containing 1 mM AEBSF and harvested with a cell scraper. The extracellular matrix fraction was isolated by sequential extraction of the cells with (1) 3% Triton X-100 in PBS containing 1 mM AEBSF; (2) 100 μg/ml DNase I in 50 mM Tris, pH 7.4, 10 mM MnCl₂, 1 M NaC, 1 mM AEBSF and (3) 2% deoxycholate in Tris, pH 8.8, 1 mM AEBSF (Wu et al., supra.) Fn in the deoxycholate insoluble extracellular matrix fraction was analyzed by immunoblot with polyclonal rabbit anti-Fn antibody MC54 and an ECL detection kit as previously described (Wu et al. (1995) J. Cell Sci. 108:821-829).

Colony formation in soft agar. ILK13-A1a3 cells that overexpress ILK (3×10⁵/well), and Ras-37 cells that overexpress H-RasVal12 (2×10³/well) were plated in 35 mm wells, in 0.3% agarose and assayed for colony growth after three weeks as described above. Fn fragments were incorporated in the agar at the final concentrations indicated.

Tumor formation in athymic nude mice. IEC-18, ILK14, or ILK13 cells were resuspended in PBS and inoculated subcutaneously into athymic nude mice (10.sup.7/mouse). Six mice were inoculated per cell line. In situ tumor formation was assessed after 3 weeks.

Tyrosine Phosphorylation of p125.sup.FAK in ILK cells. ILK13-Ala3 and ILK14-A2C3 cells growing in monolayer culture were harvested using 5 mM EDTA/PBS (Phosphate Buffered Saline, pH 7.6) and the cells were washed twice in PBS. Cells were resuspended in serum free medium and then transferred to plain tissue culture plates (Nunc), tissue culture plates precoated with 10 μg/ml Fn (Gibco/BRL) or maintained in suspension. For the suspension control cells were kept in 50 ml rocker tube. After 1 hour incubation at 37° C. in 5% CO₂ cell monolayer (for the adherent controls) and cell pellet (for the suspension controls) were washed twice in ice-cold PBS and lysed in NP-40 lysis buffer (1% NP-40; 150 mM NaCl; 50 mM Tris, pH 7.4; 1 mM EDTA, 1 mM PMSF, 0.2 U/ml aprotonin, 2 μg/ml leupeptin and 1 mM Sodium Vanadate). FAK was immunoprecipitated from 400-500 μg of total cell extract using 4 μg mouse monoclonal anti-p125.sup.FAK antibody and Protein A-Agarose conjugate (UBI). Immune complexes were washed three times in lysis buffer, boiled in SDS-PAGE sample buffer and run on a 7.5% gel. Resolved proteins were transferred to Immobilon-P (Millipore) and membrane blocked in 5% BSA (Sigma) in TBST (0.1% Tween-20 in Tris Buffered Saline, pH 7.4). Tyrosine-phosphorylated FAK was detected using the RC20H recombinant antibody (HRP-conjugate, Transduction) and ECL detection system (Amersham). Results

### Stimulation of FN Matrix Assembly by ILK

To determine whether ILK plays a role in regulation of Fn matrix assembly, the ability of cells expressing different levels of ILK to assemble a Fn matrix was analyzed. IEC-18 rat intestinal epithelial cells assembled a small amount of Fn matrix consisting of mostly short fibrils. ILK13-A1a3 cells, which were isolated from the IEC-18 cells stably transfected with a pRC/CMV expression vector containing full length ILK coding sequence, express a much higher level of ILK than the parental IEC-18 cells. The ILK overexpressing ILK13-A1a3 cells assembled an extensive Fn matrix resembling that formed by fibroblasts, whereas control transfectants (ILK14-A2C3), which express a similar level of ILK as the parental IEC-18 cells, assembled a small amount of Fn matrix that is indistinguishable from that of the IEC-18 cells fibroblasts. To exclude the possibility that the observed effect depends on a specific clone, ten additional cell lines were analyzed that were independently isolated from the cells transfected with the pRC/CMV-ILK expression vector (ILK13-A4a, A1d11, A4c, A4c3 and A4i) or the control vector (ILK14-A2C6, A2a3, A2g3, A2g8 and A3a1) Fn matrix assembly was dramatically increased in all six ILK-overexpressing cell lines (Table 3). On the other hand, all six control cell lines assembled a low level of Fn matrix resembling that of the parental IEC-18 cells. In marked contrast to overexpression of ILK, overexpression of an oncogenic H-Ras mutant in which the twelfth amino acid residue is mutated (H-RasVal12) in the IEC-18 cells abolished the assembly of Fn fibrils.

TABLE 3
Fn matrix assembly by cells expressing different levels of ILK

| | | Extracellular Fn |
|---|---|---|
| Cell Line | ILK Expression level | matrix level |
| ILK13 (A1a3, A4a, | High (wild type ILK) | High |
| A1d11, A4c) | | |
| ILK14 (A2C6, A2C3, | Low (wild type ILK) | Low |
| A2a3, A2g3, A2g8 | | |
| and A3a1), IEC-18, | | |
| MT-ILK6 (E2) | | |
| GH31RH | High (kinase- | Low |
| | inactive mutant) | |

The ILK13 cell lines were independently isolated from IEC-18 rat intestinal epithelial cells that were stably transfected with a pRC/CMV expression vector containing full length ILK coding sequence and they express a much higher level of ILK than the parental IEC-18 cells. The ILK14 cells were control transfectants. The MT-ILK1 (IIB8) cells were isolated from IEC-18 cells transfected with the sense ILK expression vector (MT-ILK1). The MT-ILK6 (E2) cells were isolated from IEC-18 cells transfected with the anti-sense ILK expression vector (MT-ILK6). The GH31R cells were isolated from IEC-18 cells transfected with a pCDNA3 expression vector encoding a ILK kinase-inactive mutant in which glutamic acid residue 359 was replaced with a lysine residue. The relative ILK expression levels were based on immunoblot analysis with anti-ILK antibodies.

To further confirm a regulatory role of ILK in Fn matrix assembly, IEC-18 cells were transfected with expression vectors containing full length ILK cDNA in the forward (sense) or the reverse (anti-sense) orientation that were under the control of metallothionein promoter (MT). The MT-ILK1 (IIB8) cells, which were derived from the IEC-18 cells transfected with the sense ILK expression vector, expressed more ILK than the MT-ILK6 (E2) cells that were derived from the IEC-18 cells transfected with the anti-sense ILK expression vector. The difference in ILK expression was maximized when the cells were grown in the presence of Zn⁺⁺ and Cd⁺⁺. Consistent with a critical role of ILK in Fn matrix assembly, the ILK overexpressing MT-ILK1 (IIB8) cells exhibit a much high Fn matrix assembly than the MT-ILK6 (E2) cells that have a much lower level of ILK. Thus, overexpression of ILK, either driven by a CMV promoter or driven by a metallothionein promoter, stimulates Fn matrix assembly.

Involvement of integrin-linked kinase activity in the cellular regulation of Fn matrix assembly. To test whether the kinase activity is involved in the stimulation of Fn matrix assembly by ILK, a kinase-inactive ILK mutant (GH31R) was overexpressed in the IEC-18 cells. Unlike cells overexpressing the wild type ILK, cells overexpressing the kinase-inactive ILK mutant did not assemble an increased amount of Fn into the extracellular matrix (FIG. 1D). Thus, the kinase activity is critical in the cellular signal transduction leading to the up-regulation of Fn matrix assembly.

Biochemical characterization of Fn matrix assembled by cells overexpressing ILK. The Fn matrix deposited by fibroblastic cells is characterized by insolubility in sodium deoxycholate. To determine whether Fn matrix induced by overexpression of ILK in the epithelial cells shares this characteristic, the cell layers were extracted with 2% sodium deoxycholate and the insoluble matrix fractions analyzed by immunoblotting. The cells overexpressing ILK (A1a3, A4a and IIB8) assembled much more Fn into the deoxycholate insoluble matrix than the cells that express relatively low level of ILK (A2C6, A2C3, and E2). By contrast, cells overexpressing H-RasVal12 failed to deposit detectable amount of Fn into the detergent insoluble matrix (H-Ras). These results are consistent with the immunofluorescent staining data. Taken together, they provide strong evidence supporting an important role of ILK in regulation of Fn matrix assembly.

Participation of the RGD containing integrin-binding domain and the amino terminal domain of Fn in ILK stimulated Fn matrix assembly. Integrin-mediated Fn matrix assembly requires at least two discrete portions of Fn, the RGD containing integrin-binding domain and the aminoterminal domain. To determine whether these domains also participate in Fn matrix assembly induced by overexpression of ILK, the 110 kDa RGD containing fragment, the 70 kDa aminoterminal domain of Fn, and an antibody against the amino terminal domain of Fn (anti-29 kDa) were utilized. Both the antibody and the Fn fragments decreased the Fn fibril formation induced by ILK. The inhibition was specific, as neither irrelevant rabbit IgG nor a 60 kDa Fn Fragment lacking the amino terminus inhibited the Fn matrix assembly. Thus, both the RGD containing integrin-binding domain and the amino terminal domain of Fn are involved in Fn matrix assembly promoted by overexpression of ILK, suggesting a role of Fn-binding integrins in this process.

Co-distribution of α₅β₁ integrin and Fn matrix in cells overexpressing ILK. To begin to identify which Fn-binding integrin mediates Fn matrix assembly induced by overexpression of ILK, cells overexpressing ILK were stained with a hamster monoclonal anti-rat α₅ integrin antibody and a rabbit polyclonal anti-Fn antibody. The double-staining experiments showed that α₅ β₁ integrin was co-localized with Fn fibrils in ILK13-A1a3 cells that overexpress ILK. In contrast, staining of the cells with an anti-rat β₃ integrin antibody revealed no distinctive staining. These results suggest that α₅ β₁ integrin, but not β₃ integrins, participate in the Fn matrix assembly induced by overexpression of ILK.

In contrast to cells that overexpress ILK, cells expressing a lower level of ILK (A2C6) have fewer clusters of α₅ β₁ integrin that could be detected by immunofluorescent staining, although these cells express the same level of cell surface α₅ β₁ integrin as the cells overexpressing ILK. Moreover, in marked contrast to ILK13-A1a3 cells that overexpress ILK, many of the structures containing α₅ β₁ integrin in the ILK14-A2C6 cells lacked detectable Fn, indicating that overexpression of ILK enhances the binding of Fn to α₅ β₁ integrin.

Effect of ILK overexpression on the formation of focal adhesion and matrix contacts. Cell adhesion to extracellular substrates is mediated by transmembrane complexes termed focal adhesions that contain integrin, vinculin and other cytoskeletal proteins. A connection between extracellular Fn and the intracellular actin cytoskeleton involving the integrin β cytoplasmic domain is required for the assembly of Fn fibrils. ILK14-A2C3 cells that express low levels of ILK formed abundant focal adhesions visualized by staining with an anti-vinculin antibody. However, only a small amount of α₅ β₁ integrin and Fn were co-localized with the focal adhesions in ILK14-A2C3 cells.

Overexpression of ILK promoted co-localization of α₅ β₁ integrin and Fn with vinculin. Thus, while cells expressing a relatively low level of ILK are not defective in the assembly of focal adhesion, a higher level of ILK promotes the assembly of complexes containing vinculin, α₅ β₁ integrin and Fn matrix. Overexpression of ILK down-regulates E-cadherin. E-cadherin is an important epithelial cell adhesion molecule mediating cell-cell interactions. Because overexpressing ILK in epithelial cells disrupted the characteristic “cobble-stone” epithelial morphology of the epithelial cells, the effect of ILK expression on the cellular level of E-cadherin was studied. The level of E-cadherin in cells expressing different amount of ILK was determined by immunoblot using an anti-E-cadherin antibody. The parental IEC-18 epithelial cells expressed abundant E-cadherin. Overexpression of H-RasVal12 in IEC-18 cells reduced the level of E-cadherin. Strikingly, E-cadherin was completely eliminated in ILK13-A1a3 and A4a cells that overexpress ILK, whereas it was present at a normal level in ILK14-A2C3 and A2C6 cells that express a similar level of ILK to the parental IEC-18 cells (FIG. 8A). These results indicate an inverse correlation between the level of ILK and that of E-cadherin.

In contrast to E-cadherin level, overexpression of ILK did not alter the ability of the cells to phosphorylate focal adhesion kinase (pp125.sup.FAK) in response to cell adhesion to Fn, indicating that tyrosine phosphorylation of pp125.sup.FAK does not transduce the signals leading to the alterations observed upon ILK overexpression, and in particular tyrosine phosphorylation of pp125.sup.FAK does not play a regulatory role in ILK induced Fn matrix assembly.

Induction of in vivo tumorigenesis by overexpression of ILK. To assess a potential role of ILK in tumorigenesis, cells expressing varying levels of ILK were injected into athymic nude mice subcutaneously. Tumors arose within three weeks in 50% to 100% of the mice injected with the ILK13 cells (10.sup.7 cells/mouse) that overexpress ILK, whereas no tumors were detected in the mice that were injected with the same number of the IEC-18 or ILK14 cells expressing lower levels of ILK (Table 4). Thus, overexpression of ILK in these epithelial cells promotes tumor formation in vivo.

TABLE 4
Tumorigenicity of ILK overexpressing IEC-18 Cells

| Cell Line | Number of Mice with Tumors at 3 weeks |
|---|---|
| IEC-18 | 0/6 |
| ILK14-A2C3 | 0/6 |
| ILK13-A1a3 | 6/6 |
| ILK13-A4a | 3/6 |

Athymic nude mice were inoculated subcutaneously with the cells expressing high (ILK13-A1a3 and A4a) or low (IEC-18 and ILK14-A2C3) levels of ILK (10⁷ cells/mouse in PBS). The mice were monitored for tumor formation at the site of inoculation after three weeks.

Inhibition of ILK induced cell growth in soft agar by amino terminal fragments of Fn that inhibit matrix assembly. One of the hallmarks of tumor forming cells is that their growth is less dependent on anchorage as measured by their ability to grow in soft agar culture. Similar to cells overexpressing H-Ras, cells overexpressing ILK were able to grow in soft agar. However, in marked contrast to the H-Ras overexpressing cells, ILK overexpressing cells assembled an abundant Fn matrix (Table 3). It was therefore tested whether the ability of the ILK overexpressing cells to grow in soft agar culture is related to the elevated level of Fn matrix assembly. The cells overexpressing ILK and the cells overexpressing H-Ras, respectively, were cultured in soft agar either in the presence or absence of the 70 kDa Fn amino terminal fragment, which inhibits the ILK induced Fn matrix assembly. The 70 kDa Fn fragment significantly inhibited the ILK induced “anchorage independent” growth in soft agar. Similar inhibition was observed with the 29 kDa fragment of Fn. In contrast, the H-Ras induced anchorage independent growth in soft agar was not inhibited by the 70 kDa Fn fragment. Moreover, the ILK induced cell growth in soft agar was not inhibited by the 60 kDa Fn Fragment that does not inhibit the Fn matrix assembly induced by ILK. These results suggest that the cell growth in soft agar induced by ILK, but not that induced by H-Ras, is at least partially mediated by a Fn matrix.

Discussion

The overexpression of ILK results in a loss of E-cadherin protein expression, offering a possible explanation for the loss of cell-cell contact in these cells. Indeed, losses of cell-cell adhesion have been implicated in tumorigenicity in vivo. ILK overexpressing cells are tumorigenic in nude mice in contrast to the parental IEC-18 intestinal epithelial cells and the control transfected clones. Thus, ILK can be considered to be a proto-oncogene. Another important finding is the apparent involvement of ILK in Fn matrix assembly. Overexpression of ILK in IEC-18 cells stimulated Fn matrix assembly. This is a property of transfected cell clones constitutively overexpressing ILK, and also of transfected clones in which ILK expression is induced using a metallothionein inducible promoter. Furthermore, Fn matrix assembly is impaired when an anti-sense ILK cDNA is induced resulting in decreased ILK expression.

The ILK-stimulated Fn matrix assembly was inhibited by the amino-terminal domain of Fn, as well as the RGD-containing integrin binding domain of Fn, suggesting that RGD-binding integrins mediate ILK functions in Fn matrix assembly. Due to the unavailability of anti-integrin function blocking antibodies against rat integrins, it has not been possible to identify directly the specific integrin(s) involved in the enhanced Fn binding and matrix assembly. However, using immunofluorescence analysis, the α₅ β₁ integrin, but not α₅ β₃, was co-localized with Fn fibrils in the ILK overexpressing cells, implicating α₅ β₁ in the matrix assembly process. Furthermore, ILK overexpression promoted the co-localization of Fn with α₅ β₁ and vinculin, whereas in the parental IEC-18 cells and control transfected cells vinculin containing focal adhesion plaques were not co-localized with Fn.

The kinase activity of ILK is clearly important in the stimulation of Fn matrix assembly, as overexpression of a kinase-inactive ILK mutant failed to enhance Fn matrix assembly. However, because ILK has potential binding sites for integrins and probably other intracellular signaling molecules, and because Fn matrix assembly can be regulated by post ligand occupancy events, it is possible that other activities of ILK may also play important roles in the stimulation of Fn matrix assembly.

Although ILK overexpressing IEC-18 cells express same levels of integrins as the parental cells, the ILK overexpressing cells gain the ability to grow in an anchorage independent manner in soft agar, and are tumorigenic in nude mice, and they organize a prolific Fn matrix. The same IEC-18 cells transfected with an activated form of H-ras, do not assemble a Fn matrix, but nevertheless are highly tumorigenic in nude mice. This represents a novel pathway of oncogenic transformation which is distinctive from H-Ras induced transformation and involves ILK and enhanced Fn matrix assembly. In fact, the ability to form a Fn matrix is important for the anchorage independent growth of transforming growth factor β (TGF β) treated fibroblasts. Fn matrix assembly also seems to be important for anchorage-independent growth in soft agar of the ILK overexpressing cells since inhibition of matrix assembly by the 29 kDa and 70 kDa amino terminal fragments of Fn, results in an inhibition in colony formation in soft agar.

The expression of activated p21^{ras} results in the disregulation of multiple signaling pathways and typically renders cells serum-independent, as well as anchorage independent for cell growth. On the other hand, the overexpression of ILK does not result in serum-independent cell growth, but induces anchorage-independent cell growth. These results indicate that ILK normally regulates adhesion-dependent signaling pathways and that the disregulation of ILK (e.g. by overexpression) induces anchorage-independent cell growth specifically. It is likely that ILK mediated signaling may be involved in the regulation of integrin inside-out signaling, as activated integrins are required for Fn matrix assembly.

The ability to assemble an extensive Fn fibrillar matrix is a property of mesenchymal cells and it is intriguing that the stimulation of this activity by ILK overexpression in the epithelial cells is accompanied by a dramatic downregulation of cellular E-cadherin expression. Numerous previous studies have established that cellular E-cadherin level or activity is downregulated during epithelial-mesenchymal transition. Moreover, in a recent study, Zuk and Hay demonstrated that inhibition of α₅ β₁ integrin, which is a substrate of ILK, significantly inhibited epithelial-mesenchymal transition of lens epithelium. It is now also widely accepted that many invasive carcinomas exhibit a loss of E-cadherin expression, and E-cadherin gene has been found to be a tumor/invasion-suppressor gene in human lobular breast cancer. The tumor suppressor gene fat in Drosophila is also homologous to cadherins. ILK may therefore be involved in coordinating cell-matrix adhesion and cell-cell adhesion in epithelial-mesenchymal transition, and overexpression of ILK may drive epithelial cells towards a mesenchymal phenotype and oncogenic transformation.

The ILK stimulated Fn matrix assembly may allow enhanced interaction of Fn with α₅ β₁. This integrin has recently been shown to be specific in supporting survival of cells on Fn, although no direct correlation was found between Fn matrix assembly and α₅ β₁ mediated cell survival. This latter conclusion was derived from the use of wild type α₅ β₁ and α₅ cytoplasmic deleted (α₅ .DELTA.Cβ₁) mutants. It is likely that for cell survival, both receptor interaction with Fn, as well as proper intracellular interactions are required. ILK overexpression in IEC-18 cells induces cell survival in suspension cultures largely due to the up-regulation of expression of cyclin D₁ and cyclin A proteins.

### EXAMPLE 10

### Expression of ILK in Human Colon Carcinoma Cells

Tumor (T) or adjacent normal (N) tissue from patients biopsied for colon carcinoma were analyzed for the expression of ILK or LEF-1 by Western blot analysis. ILK activity was further determined by an in vitro kinase assay, as described in previous examples.

These data demonstrate the strong expression of ILK in colon carcinomas, indicating an association with transformation. In accordance with the data presented in the previous example, LEF-1 expression is closely tied to ILK expression.

### EXAMPLE 11

### ILK Antisense Oligonucleotides

Antisense oligonucleotides are capable of attenuating the expression of specific genes in vivo by anealing to their RNA transcript and interfering with gene expression. As such, these oligonucleotides can be valuable tools in biological studies of gene function and well as therapeutic agents against human diseases caused by overexpression of specific genes. The technical problems facing the development of antisense oligonucleotides lie in their design. Not all antisense oligonucleotides are created equal. Due to the fact that single stranded RNA has a secondary structure consisting of self annealed regions and loops, locations on the transcript will have varying degrees of accessibility to oligonucleotides. Presently, we cannot accurately predict RNA secondary structure from its primary sequence so it is necessary to empirically test a series of antisense oligonucleotides in order to determine their accessibility and efficacy against the RNA transcript.

The following methods describe an in vitro assay designed to determine the ability of specific antisense oligonucleotides to anneal to an RNA transcript and mediate its cleavage by RNAse H. This “RNAse H” assay consists of three components: 1. an RNA transcript produced in vitro; 2. an antisense oligonucleotide, approximately 19 mer in length, complementary to a region of the RNA transcript; 3. the enzyme RNAse H, which cleaves RNA only at RNA-DNA duplexes. Cleavage is determined simply by agarose gel electrophoresis of the assay products. This assay will identify antisense oligonucleotides that have the ability to anneal to regions of the RNA transcript thus identifying these regions as having an accessible secondary structure. A series of oligonucleotides scanning the entire ILK cDNA were designed and tested using the RNAse H assay. This identified a limited number of oligonucleotides that were effective in mediating cleavage of the RNA transcript. These selected oligonucleotides were then tested for their efficacy in attenuating ILK expression in a cell culture based assay.

Results of the in vitro RNAse H assay, given in % cleavage, reflect the ability of a particular oligonucleotide sequence to bind to the target RNA transcript, and promote its RNAse H mediated cleavage. As mentioned previously, the enzyme RNAse H will only cleave at sites where the RNA is annealed to complementary DNA sequences. The oligonucleotide sequences giving high % cleavage values are expected to have a similar ability to anneal to the target RNA transcript when transfected into cells, and disrupt the expression of protein from this transcript. RNAse H is a ubiquitous enzyme in living cells and the mechanism-of-action of antisense oligonucleotides in cells and in vivo is thought to be based upon this same RNAse H mediated cleavage of RNA (Chiang et al. 1991). Thus, there is a rational basis for the use of this in vitro assay for predicting in vivo efficacy of antisense oligonucleotides. Oligonucleotides selected using the RNAse H assay, can then be analyzed for their efficacy in attentuating ILK expression in a cell culture, for example using Northern and Western blot assays. Further validation may include treatment of animals with the antisense oligonucleotide and monitoring its in vivo effects on ILK expression.

### Materials and Methods

### Solutions and Buffers

Standard procedures for controling RNAse contamination were adopted such as the use of rubber gloves, segragated ACS or molecular biology grade reagents, RNAse free prepacked pipette tips, and baked glassware (Sambrook et al. 1989).

Solutions and buffers were prepared using Millipore Milli-Q PF grade water. Diethyl pyrocarbonate (DEPC) treated water and buffers were prepared by adding diethyl pyrocarbonate (Sigma D5758) to a final concentration of 0.1%, mixing, and then incubating at 37° C. for 12 hours. The solutions were then autoclaved at 120° C. and 20 psi for 20 min. Solutions of 1 M dithiothreitol (DTT, BDH 3860), 0.5 M ethylenediaminetetraacetate disodium salt (EDTA; Sigma E5134) DEPC treated, 1 M magnesium chloride hexahydrate (Sigma M2670) DEPC treated, 3 M sodium acetate trihydrate (BDH 7610) pH 5.7 DEPC treated, 5 M sodium chloride (BDH 7710) DEPC treated, 10 M sodium hydroxide (Sigma S0899), and 1 M Tris pH 8.0 or 7.4 (BDH 9210) were prepared following Sambrook et al 1989.

One liter of 5×MOPS buffer (0.2 M MOPS pH 7.0, 50 mM sodium acetate, 5 mM EDTA) was prepared by adding 16.6 ml of 3 M sodium acetate pH 5.7 (described above) to 800 ml of Mill-Q PF water, and then adding 41.2 g of 3-(N-morpholino) propanesulfonic acid (MOPS, BDH 6310). The pH was adjusted to 7.0 using 10 M NaOH (described above). Then, 10 ml of 0.5 M EDTA pH 8.0 (described above) was added and volume was adjust to 1000 ml. This was then vacuum filter sterilized through a 0.2 micron plastic disposable filter. MOPS solutions should not be autoclaved but can be stored at room temperature wrapped in foil.

One liter of 5×TBE buffer (0.45 M Tris-borate, 0.01 M EDTA) was prepared by dissolving 54 g of Tris base (BDH 9210) and 27.5 g of boric acid (Sigma B6768) in 800 ml of Mill-Q PF water. Then 20 ml of 0.5 M EDTA DEPC treated (described above) was added and the volume adjusted to 1000 ml. Autoclaving was not required.

RNA sample buffer (66% formamide, 20% formaldehyde, and 0.65×MOPS buffer) was made by mixing 10 ml of deionized formamide (BDH 4610), 3.5 ml of 37% formaldehyde (Aldrich 25,254-9), and 2.0 ml of 5×MOPS buffer (described above). This can be stored at 20° C. for up to 6 months. The 10×RNA loading buffer (50% gylcerol, 1 mM EDTA, 0.4% xylene cyanol and 2 mg/ml of ethidium bromide was prepared my mixing 5 ml of glycerol (BDH 4750), 20 ul of 0.5 M EDTA DEPC treated (described above), 40 mg of xylene cyanol FF (BDH 9710), 2 ml of 10 mg/ml ethidium bromide solution (BDH 4410), and 3.0 ml of Milli-Q PF water.

### Preparation of DNA Oligonucleotides and RNA Transcripts

Phosphodiester oligodeoxynucleotides were produced on an Applied Biosystems 394 synthesizer using standard phosphoramidite chemistry. Antisense oligonucleotides were designed as an almost contiguous head-to-tail series, scanning the entire ILK cDNA, including its 3′ and 5′ untranslated regions.

In vitro RNA transcripts of ILK cDNA were produced with the Promega RiboMax Large Scale RNA Production System T7 and cDNA template derived from the plasmid ILK13/pRC/CMV (Hannigan et al. 1996) linearized at Xba I. The crude RNA transcript was purified using a Qiagen RNeasy mini spin kit.

### RNAse H Assay

Phosphodiester antisense oligonucleotides were diluted to 100 nM in hybridization buffer (10 mM Tris pH 7.3, 50 mM NaCl, 5 mM MgCl₂), denatured by heating to 90° C. for 2 min, and quick chilled on ice. Then, 5 μl of denatured oligonucleotide was added to wells of a GeNunc 120 μl well module (Nunc 2-32549). The purified ILK RNA transcript was diluted in hybridization buffer to a concentration of 2 μM, heated to 90° C. in a water filled aluminum heat block for 1 min, and then slowly cooled to 37° C. over a period of 30 min. Then, 5 μl of the reannealed RNA transcript was aliquoted to wells of the GeNunc module containing the antisense oligonucleotides. These were hybridized at 37° C. for 4 hr. After this, 1 μl of 10 mM DTT (described above) and 0.5 U of RNAse H (Gibco BRL 18021-014) were added to each well and further incubated at 37° C. for 20 min. The RNAse H cleavage was stopped by adding 30 μl of RNA sample buffer (described above) and 5 μl of 10×RNA loading buffer (described above). The assay products could be stored at −20° C. at this point. Prior to electrophoresis the samples were denatured by heating to 70° C. for 10 min and then cooled to room temperature. Electrophoresis was carried out on Gibco BRL Horizon 11×14 apparatus using 20 well combs, 1% agarose (BDH 2120) with 0.5 μg/ml ethidium bromide in the agarose and 0.5×TBE running buffer. The ethidium bromide stained RNA bands were visualized on a UV transilluminator using a Bio-Rad Gel Doc 1000 apparatus. Quantitation of the cleaved and uncleaved RNA bands was done using the volume analysis feature of Bio-Rad's MultiAnalyst 1.1 software, and % cleavage of RNA was calculated from the formula:

% cleaved RNA=100%×cleaved RNA/(cleaved RNA+uncleaved RNA)

Sequences with the highest % cleavage in the RNAse H assay can be expected to interfer the strongest with ILK gene expression.

### Cell Based Oligonulcleotide Transfection

Phosphorothioate antisense oligonucleotides were diluted to 100 μM in Milli-Q PF water. Transfections of oligonucleotides were carried out in a 96 well microtire plate format (Corning Cell Wells 25860) using the following parameters: 1.25 μM oligonucleotide final concentration in the media, 50% confluent IEC18-13 cells (rat intestinal cells stably overexpression ILK; Hannigan et al. 1996), (-MEM media (Gibco BRL 12000-014), and the transfection reagent Fugene 6 (Boehringer/Roche 1 814 443) used according to the directions of the manufacturer. The cells were then incubated at 37° C. and 5% CO₂ for 20 to 24 hrs before, a 3H-thymidine incorporation assay was performed on the transfected cells by adding 1 μCi of thymidine-methyl-3H (Sigma 32,222-9) to each well and incubating a further at 37° C. and 5% CO₂ for 4 hrs. The 3H-thymidine media was then removed and the cells were washed once with 100 μl per well of phosphate buffered saline pH 7.4 (PBS) and allowed to dry. Then, 100 μl of scintillation fluid was added to each well and the beta emissions were recorded using a Wallac Micro-beta counter.

ILK Anti-sense Oligonucleotides

| | | Position | | |
|---|---|---|---|---|
| | | from | Cleavage | |
| ID No. | Sequence | 5′ end | (in vitro) | Code |
| SEQ ID NO:13 | CGTCCATAGCAGCGTCCCG | ILK16345 | 62 | 0 |
| SEQ ID NO:14 | CCCGTGGTAGCAGTCGAC | ILK2811 | Na | 1 |
| SEQ ID NO:15 | CCTTCTCCGGGGAACTCCC | ILK4426 | 20 | 2 |
| SEQ ID NO:16 | CGGGACTCGGGCTGCAGGA | ILK6446 | 21 | 3 |
| SEQ ID NO:17 | GCTTTATCCTCGGGACTCG | ILK7456 | 17 | 4 |
| SEQ ID NO:18 | GGGAAGGAGGATGAACCCC | ILK9577 | 10 | 5 |
| SEQ ID NO:19 | GCCTGAGCACTGTGGAGTG | ILK11901 | 14 | 6 |
| SEQ ID NO:20 | GGGGAAGCCTGAGGACTG | ILK12508 | 19 | 7 |
| SEQ ID NO:21 | GAGTGAAAATGTCGTCCAT | ILK17557 | 59 | 8 |
| SEQ ID NO:22 | AACGGCGACTGCGTTGCCC | ILK20486 | 19 | 9 |
| SEQ ID NO:23 | TGTTGTCCAGCCACAGGCG | ILK22305 | 19 | 10 |
| SEQ ID NO:24 | TGGTTGAGGTCGTTCTCCG | ILK24224 | 17 | 11 |
| SEQ ID NO:25 | GAAGCCATGATCGTCCCCC | ILK26143 | 31 | 12 |
| SEQ ID NO:26 | CAGGCCCAGTGCAAGGGGG | ILK28163 | 19 | 13 |
| SEQ ID NO:27 | CGGCCCTCTCGGCAGGCCC | ILK29375 | 21 | 14 |
| SEQ ID NO:28 | CCACAGCAGAGCGGCCCTC | ILK30486 | 18 | 15 |
| SEQ ID NO:29 | CCGCATGATCAACATCTC | ILK32407 | 32 | 16 |
| SEQ ID NO:30 | TTGATCCGTGCCCCCCGC | ILK33821 | 26 | 17 |
| SEQ ID NO:31 | CATCCCCACGGTTCATTAC | ILK35840 | 29 | 18 |
| SEQ ID NO:32 | GGGGGTGTCATCCCCACGG | ILK36648 | 31 | 19 |
| SEQ ID NO:33 | GGCTGCCAGATGCAGGGGG | ILK38163 | 32 | 20 |
| SEQ ID NO:34 | ATATCACGGTGTCCATGAC | ILK40183 | 28 | 21 |
| SEQ ID NO:35 | ACTGCAATAGCTTCTGTAC | ILK42103 | 31 | 22 |
| SEQ ID NO:36 | CACTGCATTGATGTCTGCC | ILK44426 | 32 | 23 |
| SEQ ID NO:37 | GGGGCACATTCCCGTGTTC | ILK46648 | 34 | 24 |
| SEQ ID NO:38 | CCAAAAACAGGCATAGTGC | ILK48668 | 28 | 25 |
| SEQ ID NO:39 | GCCACTTGATCTTGGCCCC | ILK50385 | 34 | 26 |
| SEQ ID NO:40 | CATTTGCCACCAGGTCCTC | ILK52305 | 37 | 27 |
| SEQ ID NO:41 | AGATGCTGACAAGGGCCCC | ILK54123 | 36 | 28 |
| SEQ ID NO:42 | ATCTCTCCATACTTGTTAC | ILK56042 | 29 | 29 |
| SEQ ID NO:43 | CTTGGCTTTGTCCACAGGC | ILK57961 | 37 | 30 |
| SEQ ID NO:44 | AAGCTCTCTCAGGGGTGCC | ILK59779 | 37 | 31 |
| SEQ ID NO:45 | TCTCTGCCCGCTCTCGGAG | ILK61698 | 40 | 32 |
| SEQ ID NO:46 | CGGTTGAGATTCTGGCCCA | ILK63820 | 41 | 33 |
| SEQ ID NO:47 | AGAATGTGTCCTTGTATGG | ILK66143 | 43 | 34 |
| SEQ ID NO:48 | GTGCGGGTGGTCCCCTTCC | ILK68062 | 44 | 35 |
| SEQ ID NO:49 | CGGGGCCGAGTGCGGGTGG | ILK68971 | 43 | 36 |
| SEQ ID NO:50 | GGGTTCCATTTCGGGGCCG | ILK70082 | 39 | 37 |
| SEQ ID NO:51 | CCAGAGTGTTTGTTCAGGG | ILK71698 | 39 | 38 |
| SEQ ID NO:52 | AGTTAAGCTGTTTGAAGTC | ILK73921 | 46 | 39 |
| SEQ ID NO:53 | CTCGTTGAGCTTCGTCAGG | ILK75941 | 36 | 40 |
| SEQ ID NO:54 | TCCATAGCTCTCCAGAGTG | ILK78163 | 46 | 41 |
| SEQ ID NO:55 | GCCCTGCCAGCGGCCCTTC | ILK79880 | 46 | 42 |
| SEQ ID NO:56 | GCACCTTCACGACAATGTC | ILK80220 | 55 | 43 |
| SEQ ID NO:57 | ACTCCAGTCTCGAACCTTC | ILK84022 | 46 | 44 |
| SEQ ID NO:58 | AAGTCCCTGCTCTTCCTTG | ILK86042 | 50 | 45 |
| SEQ ID NO:59 | GCCGGGGACACTCTTCATTG | ILK88061 | 56 | 46 |
| SEQ ID NO:60 | TCCTGAGCCGGGGACACTC | ILK88668 | 46 | 47 |
| SEQ ID NO:61 | GAGCACATTTGGATGCCAG | ILK90991 | 47 | 48 |
| SEQ ID NO:62 | GGCAGGCACCTAGCACTGG | ILK92810 | 36 | 49 |
| SEQ ID NO:63 | ATGAGGAGCAGGTGGAGAC | ILK94830 | 38 | 50 |
| SEQ ID NO:64 | AGTGTGTGATGAGAGTAGG | ILK96749 | 54 | 51 |
| SEQ ID NO:65 | AGGGATCCATACGGCATCC | ILK98668 | 42 | 52 |
| SEQ ID NO:66 | TTCATGTAGTACATTGTAG | ILK100587 | 44 | 53 |
| SEQ ID NO:67 | CCACGACGAAATTGGTGCC | ILK102406 | 41 | 54 |
| SEQ ID NO:68 | TTCACAGCCTGGCTCTGGT | ILK104325 | 45 | 55 |
| SEQ ID NO:69 | TGCCATGTCCAAAGCAAAC | ILK106244 | 40 | 56 |
| SEQ ID NO:70 | GTAGGAAGGCCATGCCCCT | ILK108163 | 43 | 57 |
| SEQ ID NO:71 | ATGAGGGGCTCTAGTGTGT | ILK110082 | 41 | 58 |
| SEQ ID NO:72 | ATTGAGTGCATGTCGTGGG | ILK111901 | 43 | 59 |
| SEQ ID NO:73 | CAATCATTACACTACGGCT | ILK113820 | 38 | 60 |
| SEQ ID NO:74 | TTCGGGCAGTCATGTCCTC | ILK115941 | 32 | 61 |
| SEQ ID NO:75 | AACTTGACATCAGCCATGC | ILK118163 | 33 | 62 |
| SEQ ID NO:76 | ACCAGGACATTGGAAAGAG | ILK120082 | 34 | 63 |
| SEQ ID NO:77 | AGGCAGGTGCATACATGCG | ILK121901 | 30 | 64 |
| SEQ ID NO:78 | CGGGGGCTACCCAGGCAGG | ILK123113 | 24 | 65 |
| SEQ ID NO:79 | AGAGCTTCGGGGGCTACCC | ILK123820 | 22 | 66 |
| SEQ ID NO:80 | GTCTTCAGGCTTCTTCTGC | ILK125739 | 27 | 67 |
| SEQ ID NO:81 | TCTGCTGAGCGTCTGTTTG | ILK127759 | 30 | 68 |
| SEQ ID NO:82 | GCACTGCAAAACTCCACATG | ILK129778 | 36 | 69 |
| SEQ ID NO:83 | GTGTCACCAGTTCCCACAG | ILK131800 | 33 | 70 |
| SEQ ID NO:84 | GGGTACCTCCCGTGTCACC | ILK132911 | 34 | 71 |
| SEQ ID NO:85 | CCATATTGGAGAGGTCAGC | ILK135133 | 26 | 72 |
| SEQ ID NO:86 | GGCCTTCCAATGCCACCTT | ILK138163 | 26 | 73 |
| SEQ ID NO:87 | GGTGGGATGGTAGGCCGAA | ILK140082 | 35 | 74 |
| SEQ ID NO:88 | CACATGAGGGGAAATACCT | ILK141901 | 41 | 75 |
| SEQ ID NO:89 | CATGCAGATCTTCATGAGC | ILK144325 | 41 | 76 |
| SEQ ID NO:90 | GGGTCGCTTTGCAGGGTCT | ILK146749 | 42 | 77 |
| SEQ ID NO:91 | GGATAGGCACAATCATGTC | ILK149274 | 29 | 78 |
| SEQ ID NO:92 | ACTTGTCCTGCATCTTCTC | ILK151395 | 37 | 79 |
| SEQ ID NO:93 | GGCAAGGACCTTCCAGTCC | ILK153315 | 30 | 80 |
| SEQ ID NO:94 | CCCGACACCTCTGGAGTTC | ILK155335 | 27 | 81 |
| SEQ ID NO:95 | GTGCATTCCCCCAACCATG | ILK157355 | 36 | 82 |
| SEQ ID NO:96 | GGGGAGGTGCATTCCCCC | ILK157962 | 35 | 83 |
| SEQ ID NO:97 | GGCCTGCTGCTTTGGGGAGG | ILK159273 | 50 | 84 |
| SEQ ID NO:98 | GCAACCAGAGGCCTGCTGC | ILK160183 | 50 | 85 |
| SEQ ID NO:99 | GGAGGCGGGGGAGGCAACC | ILK161496 | 43 | 86 |
| SEQ ID NO:100 | CCATGACTGGAGGCGGGGG | ILK162204 | 41 | 87 |
| SEQ ID NO:101 | ATGGACCCCAGGCTGGGGT | ILK164527 | 36 | 88 |
| SEQ ID NO:102 | GGGGGAAGGGGATGGACCCC | ILK165637 | 27 | 89 |
| SEQ ID NO:103 | GGTAGGGATGGGGGAAGGGG | ILK166546 | 26 | 90 |
| SEQ ID NO:104 | CCCGCCCCTCTTGCGCACAG | ILK168667 | 37 | 91 |
| SEQ ID NO:105 | GCTCTGAGCCCGCCCCTC | ILK169477 | 35 | 92 |
| SEQ ID NO:106 | GACACCATGTGGCAAGTGAC | ILK171798 | 43 | 93 |
| SEQ ID NO:107 | GCTGATCCCTCCCATGTTGG | ILK173920 | 53 | 94 |
| SEQ ID NO:108 | GGCCGGGCTGATCCCTCCC | ILK174527 | 45 | 95 |
| SEQ ID NO:109 | TAATAAACTTTATTGTGAC | ILK176547 | Na | 96 |

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

What is claimed is:

1. An oligonucleotide comprising at least 18 nucleotides, wherein the sequence is complementary to an mRNA encoding human integrin-linked kinase.

2. The oligonucleotide according to claim 1, wherein said sequence is complementary to SEQ ID NO:1.

3. The oligonucleotide according to claim 2, wherein said sequence is selected from the group consisting of SEQ NO:13 to SEQ ID NO:109.

4. The oligonucleotide according to claim 3; and a pharmaceutically acceptable carrier.

5. A method of inhibiting expression of integrin linked kinase, the method comprising:
administering an oligonucleotide composition comprising an oligonucleotide comprising at least 18 nucleotides, wherein the sequence is complementary to an mRNA encoding human integrin linked kinase; and a pharmaceutically acceptable carrier.

6. The method according to claim 5, wherein said sequence is complementary to SEQ ID NO:1.

7. The method according to claim 6, wherein said sequence is selected from the group consisting of SEQ NO:13 to SEQ ID NO:109.
